(19) United States
(12) Patent Application Publication
(10) Pub. No.: US 2006/0045912 A1
(43) Pub. Date: Mar. 2, 2006

(54) 4-phenylbutyric acid controlled-release formulations for therapeutic use

(76) Inventor: Peter Truog, Chur (CH)

Correspondence Address: KING & SPALDING LLP, 191 PEACHTREE STREET, N.E., 45TH FLOOR, ATLANTA, GA, 30303-1763, US

(21) Appl. No.: 11/215,576

(22) Filed: Aug. 30, 2005

Related U.S. Application Data
(60) Provisional application No. 60/605,696, filed on Aug. 30, 2004.

(51) Int. Cl. A61K 9/22; A61K 31/19

(52) U.S. Cl. 424/468; 514/570

(57) ABSTRACT

Controlled-release formulations and dosage forms containing 4-phenylbutyric acid sodium salt, or other pharmaceutically acceptable salts, esters or prodrugs, and a controlled release material for use in the treatment of diseases and disorders including neoplastic disorders and neurodegenerative diseases The formulations provide extended release and extended half-life.

### CROSS REFERENCE TO RELATED APPLICATIONS

This present application claims priority to U.S. Provisional Patent Application No. 60/605,696, filed on Aug. 30, 2004, the contents of all of which are incorporated herein by reference.

### FIELD OF THE INVENTION

The invention provides controlled-release pharmaceutical formulations of 4-phenylbutyric acid and its pharmaceutically acceptable salts, solvates, esters and prodrugs for therapeutic use. Controlled-release 4-phenylbutyric acid formulations are useful in the treatment of various disorders, including cancer (e.g., prostate cancer) and neurodegenerative diseases (e.g., spinal muscular atrophy, amyotrophic lateral sclerosis).

### BACKGROUND OF THE INVENTION

Sodium phenylbutyrate (4-phenylbutyric acid, sodium salt) (4-PBA) is a low molecular weight aromatic carboxylic acid. In 1987, it was approved for use in the United States for the treatment of urea cycle disorders in children (Brusilow S W, Maestri N E. “Urea cycle disorders: Diagnosis, pathophysiology, and therapy” Adv. Pediatr. (1996) 43:127-170). 4-PBA functions as an ammonia scavenger in the treatment of urea cycle disorders, clearing the toxic substance from the body. It is classified as an Orphan Drug, and manufactured in a 500 mg tablet form (Buphenyl®; Medicis).

International patent application No. WO 85/04805 by Brusilow et al. describes a process for waste nitrogen removal in humans, wherein 4-phenylbutyrate is administered in a therapeutically effective dose. See also U.S. Pat. No. 4,457,942.

4-PBA has also been studied as a potential treatment for a variety of other disorders. The anti-cancer effects of 4-PBA have been evaluated in Phase I/II clinical trials (Gilbert J. et al., “A phase I dose escalation and bioavailability study of oral sodium phenylbutyrate in patients with refractory solid tumor malignancies Clin Cancer Res. (2001) (8):2292-300); Gore S D, et al., “Impact of the putative differentiating agent sodium phenylbutyrate on myelodysplastic syndromes and acute myeloid leukemia,” Clin Cancer Res. (2001) 7(8):2330-9; Chang SM et al., “Phase II study of Phenylacetate in patients with recurrent malignant glimoa: a North American Brain Tumor Consortium report”. J. Clin. Oncol., (1999) 17:984-990; and Sung M W and Waxman S. “Chemodifferentiation Therapy with Fluorouracil (EU) and Phenylbutyrate (PB) in Advanced Colorectal Cancer: A Phase I Trial” Proceedings of the AACR (1999).

4-PBA has also been evaluated as a treatment for certain blood disorders (Fibach E, Prasanna P, Rodgers G P, Samid D. “Enhanced fetal hemoglobin production by phenylacetate and. 4-phenylbutyrate in erythroid precursors derived from normal donors and patients with sickle cell anemia and beta-thalassemia” Blood. (1993) 82(7):2203-9; Resar L M et al., “Induction of fetal hemoglobin synthesis in children with sickle cell anemia on low-dose oral sodium phenylbutyrate therapy” J Pediatr Hematol Oncol. (2002) 24(9):737-41; MacMillan et al., “Treatment of Two Infants with Cooley's Anemia with Sodium Phenylbutyrate” Ann N Y Acad. Sci. (1998) 850:452-4; and Collins AF et al., “Oral sodium phenylbutyrate therapy in homozygous beta thalassemia: a clinical trial” Blood (1995) 85: 4349).

U.S. Pat. Nos. 5,661,179; 5,710,178, and 5,654,333 to Samid et al. disclose compositions and methods using phenylacetic acid derivatives for both cancer therapy and prevention of a number of pathologies, including anemia, severe β-chain hemoglobinopathies, and AIDS.

U.S. Patent Application No. 2004/0024067 by Remiszewski et al. discloses hydroxamate compounds which are deacetylase inhibitors said to have anti-proliferative properties. Butyric acid and its derivatives, including sodium phenylbutyrate, are described as histone deacetylase inhibitors and noted to induce apoptosis in vitro in human colon carcinoma, leukemia and retinoblastoma cell lines.

U.S. Patent Application No. 2004/0142859 by Steffan et al. discloses methods for treating a variety of diseases and disorders, including polyglutamine expansion diseases such as Huntington's disease, neurological degeneration, psychiatric disorders, and protein aggregation disorders and diseases, by administering to patients in need thereof of a therapeutically effective amount of one or more deacetylase inhibitors. Sodium phenylbutyrate is disclosed as a deacetylase inhibitor. Diseases of neurological degeneration are also disclosed, e.g., Kennedy's disease (spinobulbar muscular atrophy).

European patent application No. EP 1 206 936 A2 describes pharmaceutical phenylacetic acid compositions, including 4-phenylbutyric acid sodium salt compositions, for treating or preventing hypercholesterolemia and hypertriglyceridemia, as well as reducing patient LDL cholesterol levels and the probability of advanced stage atherosclerosis and associated instances such as angina, stroke, or heart attack.

The clinical use of 4-phenylbutyric acid sodium salt has been hampered by the compound's short physiological half-life. 4-PBA breaks down quickly within the body to phenylacetic acid and is rapidly eliminated from cells and excreted. As a result, very high concentrations are required in order to achieve therapeutic effects (e.g., up to 50 grams a day). Therapeutic use of such large amounts of 4-PBA is problematic for several reasons, including high cost, the need to continue therapy for months or years, and patient compliance issue (i.e., 50 grams is equal to 100 tablets per day).

U.S. Pat. No. 6,207,195 to Walsh, et al., describes the use of 4-phenylbutyrate-containing nanospheres for the treatment of cystic fibrosis using CFTR gene therapy and other disorders including tumors, urea cycle disorders and certain blood disorders. According to the specification, the nanosphere formulations of 4-phenylbutyrate are formed by complexing gelatin or other polymeric cations similar to gelatin with nucleic acids so as to form nanoparticles by a cross-linking reaction. The polymeric cation is said to have a molecular weight of between 19,000 and 30,000, and poly-1-lysine and chitosan are particular preferred. The nanoparticles can be administered in a variety of manners, and are said to allow for lower dosages of 4-phenylbutyric acid. Desirable dosages are said to range from 10 to 100 μg per day, in single or divided doses. It is noted that dosages in the range of 1 μg to 20 mg can be used. See also WO 98/56370 and WO 00/18294 (Johns Hopkins University).

International patent application No. WO 00/56153 by Chaturvedi, et al. (Vertex Pharmaceuticals), describes orally available low-dose butyrate and butyrate analogue compositions and methods for use in ameliorating β-hemoglobinopathies, cystic fibrosis, and cancer. According to the specification, compositions of an orally available butyrate prodrug or salt and a pharmaceutically acceptable carrier can be prepared which allow for the production of a serum butyrate blood concentration of 10-200 μM for 1 to 8 hours. For butyrate prodrug compounds which release 1 mole of butyrate or butyrate analog per mole of compound, the amount is 500 mg to 10 grams. The application is also directed to a method of treating a disease which involves treating the patient each day for 2 to 6 days with a prodrug, salt or analog of butyrate sufficient to maintain that serum butyrate blood concentration, and then halting such treatment for a period of 15 to 30 consecutive days before reinitiating such treatment.

European patent application No. EP 1 232 746A1 by ForTe Beheer, B. V. describes a suspendible dry powder mixture composition containing a pharmacologically active substance, a gellant or thickener, and at least one xanthan gum having a specific particle size distribution. This dry powder mixture composition is for use in the preparation of a suspension of the active substance in a liquid, thereby allowing for the formation of liquid or semi-liquid pharmaceutical compositions. These liquid compositions are preferably administered orally, such as by drinking. A commercial embodiment of this technology is an oral dose powder packet of sodium phenylbutyrate marketed by ForTe Beheer B V (The Netherlands).

Several other studies have examined the pharmacokinetics of butyric acid salts and derivatives having a variety of prodrug modifications. Desmet, G. et al. examined the excretion profile of butyric acid xylitol esters and found that such prodrugs had a slow excretion rate and reduced systemic metabolism (Eur. J. Drug. Metabol. Pharmacokinet (1991) 3: 348-351). Several other prodrug esters of butyric acid and butyric acid derivatives such as 4-phenylbutyrate have been investigated, with varying results, including esters derived from monosaccharides (Pouillart, P., et al., Int. J. Cancer (1991) 49: 89-95; Pouillart, P., et al., J. Pharm. Sci. (1992) 81: 241-244) and cholesterol, such as cholesteryl-butyrate in solid lipid nanospheres (Pellizzaro, C., Anticancer Res. (1999) 19: 3921-3925) butyric triglycerides (Planchon, P., et al., J. Pharm. Sci. (1993) 82: 1046-1048) and Zn²⁺-chelating, motif-tethered, short-chain fatty acids of phenylbutyrate (Lu, Q., et al., J. Med. Chem. (2004) 47: 467-474).

International patent publication No. WO 03/022253A1 by Lunamed AG describes a pharmaceutical unit dosing form that contain a therapeutically effective dose of a 4-phenylbutyric acid salt having prolonged release of the active ingredient, and suitable for alleviating and curing various diseases, especially cancer, upon once or twice daily oral administration. The prolonged release dosage form is a matrix of hydroxypropylmethylcellulose (HPMC) and 4-phenylbutyric acid salt, as well as other pharmaceutically acceptable excipients. See also U.S. Patent Application No. 2004/0180962.

U.S. Pat. No. 6,403,646 by Perlmutter discloses a method for the treatment of alpha-1-antitrypsin deficiency caused by the protease inhibitor type Z mutation by administration of phenylbutyric acid derivatives. The specification states that solid dosage forms for oral administration (e.g., capsules, tablets, pills, powders, and granules) can contain a controlled-release formulation as can be provided in a dispersion of active compound in hydroxypropylmethyl cellulose. See also U.S. Pat. No. 5,939,455 and U.S. Patent Application No. 2003/0195256.

It is an object of the present invention to provide new controlled-release compositions of 4-phenylbutyric acid and methods of using such controlled-release compositions for treating diseases or disorders in a patient.

It is another object of the present invention to provide new controlled-release compositions and methods which permit a reduction in total dose of 4-phenylbutryric acid in comparison to standard therapy.

It is a further object of the present invention to provide new controlled-release compositions and methods which reduce fluctuations in 4-phenylbutyric acid blood levels.

It is another object of the invention to provide new controlled-release compositions and methods which provide therapeutic blood levels of 4-phenylbutyric acid over an extended time period.

It is yet another object to provide new controlled-release compositions and methods which substantially reduce the cost of conventional 4-phenylbutryic acid therapy.

It is another object of the present invention to provide new controlled-release compositions of 4-phenylbutyric acid and methods of using such controlled-release compositions which increase patient compliance with therapy.

It is a particular object of the present invention to provide new-controlled release formulations of 4-phenylbutyric acid for the treatment of cancer and neurodegenerative disorders.

### SUMMARY OF THE INVENTION

The present invention is a new controlled-release formulation of 4-phenylbutyric acid and pharmaceutically acceptable salts, solvates, esters and prodrugs thereof. The invention further includes methods for using controlled-release formulations of 4-phenylbutyric acid and pharmaceutically acceptable salts, solvates, esters and prodrugs thereof in therapeutic applications, including treatment of cancer (e.g., prostate cancer) and neurodegenerative diseases (e.g., spinal muscular atrophy, amyotrophic lateral sclerosis). The present invention permits lower therapeutically-effective amounts of the 4-phenylbutyric acid which maintain the desired therapeutic effect over a period of time, and the number of administrations per day is lowered so as to increase patient compliance and reduce the cost of therapy.

Accordingly, one aspect of the present invention is a controlled-release composition including a therapeutically effective amount of 4-phenylbutyric acid, or pharmaceutically acceptable salts, esters or prodrugs thereof, dispersed in a polymer matrix, wherein the polymer matrix comprises a co-polymer or terpolymer. In one embodiment of the present invention, the co-polymer or terpolymer is a hydroxypropylmethylcellulose co-polymer or terpolymer.

In one embodiment, the present invention is a controlled-release composition including a therapeutically effective amount of 4-phenylbutyric acid, or pharmaceutically acceptable salts, esters or prodrugs thereof, dispersed in a polymer matrix, wherein the polymer matrix comprises a polymer blend.

The polymers included in the polymer blend can vary. In one embodiment, the polymer blend includes at least one hydrophilic polymer. In another particular embodiment, the polymer blend includes two or more hydrophilic polymers. Representative, non-limiting hydrophilic polymers include cellulose derivatives (e.g., cellulose ethers), non-cellulose polysaccharides, polyethylene oxide, polyvinyl alcohols and acrylic acid co-polymers.

In a particular embodiment, the present invention is a controlled-release composition including a therapeutically effective amount of 4-phenylbutyric acid, or pharmaceutically acceptable salts, esters or prodrugs thereof, dispersed in a polymer matrix, wherein the polymer matrix comprises a blend of (i) hydroxypropylmethylcellulose; and (ii) methylcellulose, hydroxyethylcellulose, hydroxymethylcellulose, carboxy-methylcellulose, ethylcellulose, hydroxpropylcellulose or microcrystalline cellulose.

In another embodiment, the polymer blend includes a hydrophilic polymer (e.g., hydroxypropylmethylcellulose) in combination with a non-hydrophilic polymer, such as a hydrophobic polymer.

Another aspect of the present invention provides a controlled release composition including therapeutically effective amount of 4-phenylbutyric acid, or a pharmaceutically acceptable salt, ester or prodrug thereof, dispersed in a polymer matrix, wherein the polymer matrix includes at least one cellulose ether polymer selected from the group consisting of methylcellulose, hydroxyethyl cellulose and hydroxypropyl cellulose.

A still further aspect of the present invention provides a controlled release composition including a therapeutically effective amount of 4-phenylbutyric acid, or a pharmaceutically acceptable salt, ester or prodrug thereof, dispersed in a polymer matrix, wherein the polymer matrix includes at least one hydrophilic polymer selected from the group consisting of non-cellulose polysaccharides, polyethylene oxide, polyvinyl alcohols and acrylic acid co-polymers.

The 4-phenylbutyric salts are suitable for use in the controlled release formulations of the present invention. 4-phenylbutyric acid salts may vary, and include, for example, alkali metal salts, ammonium salts, substituted ammonium salts, or acid addition salts. In a preferred embodiment, the 4-phenylbutyric acid salt is a sodium salt, or sodium phenylbutyrate.

In one embodiment of the present invention, the controlled release composition may also include a second therapeutic agent in addition to 4-phenylbutyric acid. Suitable second therapeutic agents include, but are not limited to, chemotherapeutic agents and agents used to treat neurodegenerative diseases. Representative, non-limiting chemotherapeutic agents include alkylating agents, antimetabolites, plant alkaloids, topoisomerase inhibitors, anti-tumor antibiotics and hormonal agents. Agents used to treat neurodegenerative diseases include, for example, 2-amino-6-(trifluoromethoxy) benzothiazole, valproic acid, suberoylanilide hydroxamic acid, hydroxyurea, aclarubicin, quanzolines, tetracycline derivatives, aminoglycosides, indoprofen, creatine, riluzole and carnitine.

Another aspect of the invention provides a method of treating a disease or disorder in a subject in need thereof (e.g., a human) using the controlled release composition of the present invention.

In one embodiment, the present invention provides a method of treating a disease or disorder by administering to a subject in need thereof a controlled release composition including a therapeutically effective amount of 4-phenylbutyric acid, or a pharmaceutically acceptable salt, ester or prodrug thereof, dispersed in a polymer matrix, wherein the polymer matrix includes a co-polymer, terpolymer or polymer blend. In a particular embodiment of the method, the co-polymer, terpolymer or polymer blend includes hydroxypropylmethylcellulose.

In another embodiment, the present invention provides a method of treating a disease or disorder by administering to a subject in need thereof a controlled release composition including a therapeutically effective amount of 4-phenylbutyric acid, or a pharmaceutically acceptable salt, ester or prodrug thereof, dispersed in a hydrophilic polymer matrix, wherein the hydrophilic polymer is a cellulose derivative selected from the group consisting of methylcellulose, hydroxyethyl cellulose, hydroxypropyl cellulose, carboxymethylcellulose, hydroxomethylcellulose, hemicellulose, and methylcellulose.

In a further embodiment, the present invention provides a method of treating a disease or disorder by administering to a subject in need thereof a controlled release composition containing a therapeutically effective amount of 4-phenylbutyric acid, or a pharmaceutically acceptable salt, ester or prodrug thereof, dispersed in a hydrophilic polymer matrix, wherein the hydrophilic polymer is selected from the group consisting of non-cellulose polysaccharides, polyethylene oxide, polyvinyl alcohols and acrylic acid co-polymers.

The method of the present invention can be used to treat a variety of diseases and disorders including, but not limited to, neoplastic disorders (e.g., prostate cancer), neurodegenerative disorders (e.g., spinal muscular atrophy, amyotrophic lateral sclerosis), urea cycle disorders, hematological disorders, infectious diseases, cystic fibrosis, and protein localization or aggregation disorders.

In one embodiment of the method, the controlled release composition further includes a second therapeutic agent in addition to 4-PBA. The second therapeutic agent may include, for example, a chemotherapeutic agent or agent used to treat a neurodegenerative disease.

In a particular embodiment of the method, the controlled release composition contains 4-phenylbutyric acid sodium salt (i.e., sodium phenylbutyrate).

In a preferred embodiment, the present invention provides a method of treating spinal muscular atrophy by administering to a subject in need thereof a controlled release composition including a therapeutically effective amount of 4-phenylbutyric acid, or a pharmaceutically acceptable salt, ester or prodrug thereof, dispersed in a polymer matrix. In one embodiment, the polymer matrix is a hydrophilic polymer matrix. The hydrophilic polymer may be, for example, a cellulose derivative such as a cellulose ether, non-cellulose polysaccharide, polyethylene oxide, polyvinyl alcohol or acrylic acid co-polymers. In a preferred embodiment, the polymer matrix is a hydroxymethylcellulose matrix. The controlled release composition may optionally include a second therapeutic agent, such as valproic acid, suberoylanilide hydroxamic acid, hydroxyurea, aclarubicin, quanzolines, tetracycline derivatives, aminoglycosides, indoprofen, creatine, riluzole and carnitine. In a preferred embodiment, the 4-phenylbutyric acid salt is sodium phenylbutyrate.

In another preferred embodiment, the present invention provides a method of treating amyotrophic lateral sclerosis by administering to a subject in need thereof a controlled release composition including a therapeutically effective amount of 4-phenylbutyric acid, or a pharmaceutically acceptable salt, ester or prodrug thereof, dispersed in a polymer matrix. The polymer matrix may be, for example, a hydrophilic polymer matrix. Representative, non-limiting hydrophilic polymers suitable for use in the hydrophilic polymer matrix include cellulose derivatives, non-cellulose polysaccharides, polyethylene oxide, polyvinyl alcohols and acrylic acid co-polymers. In a preferred embodiment, the hydrophilic polymer matrix is an hydroxypropylmethylcellulose matrix. Optionally, the controlled release composition may include a second therapeutic agent, such as 2-amino-6-(trifluoromethoxy) benzothiazole. In a preferred embodiment, the 4-phenylbutyric acid sodium salt is sodium phenylbutyrate

In yet another preferred embodiment, the present invention provides a method of treating a neoplastic disease or disorder by administering to a subject in need thereof a controlled release composition including a therapeutically effective amount of 4-phenylbutyric acid, or a pharmaceutically acceptable salt, ester or prodrug thereof, dispersed in a polymer matrix, wherein the polymer matrix includes a co-polymer, terpolymer or polymer blend. In a particular embodiment, the polymer matrix is a polymer blend incorporating hydroxpropylmethylcellulose.

In a further preferred embodiment, the present invention provides a method of treating a neoplastic disease or disorder by administering to a subject in need thereof a controlled release composition including a therapeutically effective amount of 4-phenylbutyric acid, or a pharmaceutically acceptable salt, ester or prodrug thereof, dispersed in a hydrophilic polymer matrix, wherein the hydrophilic polymer is cellulose ether selected from the group consisting of methylcellulose, hydroxpropylmethylcellulose, hydroxyethyl cellulose and hydroxylpropyl cellulose.

In still another preferred embodiment, the present invention provides a method of treating a neoplastic disease or disorder by administering to a subject in need thereof a controlled release composition including a therapeutically effective amount of 4-phenylbutyric acid, or a pharmaceutically acceptable salt, ester or prodrug thereof, dispersed in a hydrophilic polymer matrix, wherein the hydrophilic polymer is selected from the group consisting of non-cellulose polysaccharides, polyethylene oxide, polyvinyl alcohols and acrylic acid co-polymers

In one embodiment, the method of the present invention is used to treat a neoplastic disorder in a human. The neoplastic disorder may be, for example, bladder cancer, breast cancer, colon cancer, endometrial cancer, kidney cancer, leukemia, lung cancer, melanoma, non-Hodgkin's lymphoma, pancreatic cancer, prostate cancer, skin cancer or thyroid cancer. In a preferred embodiment, the neoplastic disorder is prostate cancer.

Optionally, the method of the present invention involves administration a controlled release composition that further includes a second therapeutic agent useful in the treatment of neoplastic disease (e.g., prostate cancer). Non-limiting examples of second therapeutic agents include chemotherapeutic agents, luteinizing hormone releasing hormone (LH-RH) agonists and anti-androgen agents.

### DETAILED DESCRIPTION OF THE INVENTION

The invention describes controlled-release formulations of 4-phenylbutyric acid, and its pharmaceutically acceptable salts, esters, and prodrugs, having a prolonged half-life. The controlled-release formulations are useful in the treatment of various disorders in a subject, including neoplastic disorders (e.g., prostate cancer) and neurodegenerative diseases (e.g., SMA, ALS).

The controlled-release pharmaceutical composition contains at least 4-phenylbutyric acid, or a pharmaceutically acceptable salt, ester, or prodrug thereof and at least one release-rate modifier (e.g., a polymer). The composition may optionally include one or more pharmaceutically acceptable carriers, excipients or diluents. In one embodiment, the composition may contain one or more therapeutic agents in addition to 4-PBA.

A. Definitions

The term “therapeutically effective dose” refers to that amount of the compound that results in achieving the desired effect. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio of LD₅₀ to ED₅₀. Compounds that exhibit high therapeutic indices (i.e., a toxic dose that is substantially higher than the effective dose) are preferred. The data obtained can be used in formulating a dosage range for use in humans. The dosage of such compounds preferably lies within a range of circulating concentrations that include the ED₅₀ with little or no toxicity. The dosage can vary within this range depending upon the dosage form employed, and the route of administration utilized.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “release rate modifier” refers to a substance or structure which will modify the rate of release of the therapeutic agent from the pharmaceutical formulation according to the invention. The release rate modifier will assist in providing a controlled release of the therapeutic agent and can cooperate with other components in the formulation to provide either a delayed, sustained, timed, pH dependent, targeted, or further controlled delivery of the therapeutic agent.

As used herein, “controlled-release” refers to release of the therapeutically active agent from the formulation at a controlled rate such that therapeutically beneficial blood levels (but below toxic levels) of the medicament are maintained over an extended period of time, e.g., providing a 6 hour, 12 hour or a 24 hour dosage form.

The term “prodrug” as used herein refers to compounds that are transformed in vivo to a compound of the present invention, for example, by hydrolysis. Prodrug design is discussed generally in Hardma et al. (Eds.), Goodman and Gilman's The Pharmacological Basis of Therapeutics, 9th ed., pp. 11-16 (1996). Another discussion is also provided by Higuchi, et al., in Prodrugs as Novel Delivery Systems, Vol. 14. ASCD Symposium Series, and in Roche (ed.), Bioreversible Carriers in Drug Design American Pharmaceutical Association and Pergamon Press (1987). Typically, administration of a drug is followed by elimination from the body or some biotransformation whereby the biological activity of the drug is reduced or eliminated. Alternatively, a biotransformation process can lead to a metabolic by-product that is more or equally active compared to the drug initially administered. Increased understanding of these biotransformation processes permits the design of so-called “prodrugs,” which, following a biotransformation, become more physiologically active in their altered state. Prodrugs, therefore, as used within the scope of the present disclosure, encompass compounds that are converted by some means to pharmacologically active metabolites.

The term “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art.

The term “unit dosage form” is used herein to mean a single or multiple dose form containing a quantity of the active ingredient and the diluent or carrier, said quantity being such that one or more predetermined units are normally required for a single therapeutic administration. In the case of multiple dose forms, such as liquids or scored tablets, said predetermined unit will be one fraction such as a half or quarter of a scored tablet of the multiple dose form. It will be understood that the specific dose level for any patient will depend upon a variety of factors including the indication being treated, the therapeutic agent employed, the activity of the therapeutic agent, severity of the indication, patient health, age, sex, weight, diet, and pharmacologic response, the specific dosage form employed and other such factors.

The term “subject”, as used herein, refers to a cell or organism that exhibits the properties associated with the non-cancerous diseases or disorders described herein. The subjects are typically vertebrates, preferably mammals. It is preferred that the mammal, as a subject or patient in the present disclosure, is from the family of Primates, Carnivora, Proboscidea, Perissodactyla, Artiodactyla, Rodentia, and Lagomorpha. It is even more preferable that the mammal vertebrate of the present invention be Canis familiaris (dog), Felis catus (cat), Elephas maximus (elephant), Equus caballus (horse), Sus domesticus (pig), Camelus dromedarious (camel), Cervus axis (deer), Giraffa camelopardalis (giraffe), Bos taurus (cattle/cows), Capra hircus (goat), Ovis aries (sheep), Mus musculus (mouse), Lepus brachyurus (rabbit), Mesocricetus auratus (hamster), Cavia porcellus (guinea pig), Meriones unguiculatus (gerbil), and Homo sapiens (human). Most preferably, the subject or patient as used within the present invention is Homo sapiens (human).

B. Compounds

The pharmaceutical compositions may include 4-phenylbutyric acid (I), and its pharmaceutically acceptable salts (e.g., X is a cation, including, but not limited to, an alkali metal cation such as sodium or potassium, an ammonium, or a substituted ammonium), esters, and prodrugs.

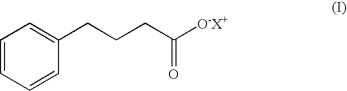

4-Phenylbutyric acid and pharmaceutically acceptable salts, esters and produgs, can be prepared according to methods known in the art, can be isolated from body fluids or obtained from commercial sources. In one embodiment, 4-phenylbutyrate and 4-phenylbutyrate derivatives can be prepared by the Arndt-Einstert reaction, using diazomethane with silver oxide and sodium thiosulfate, as described by Jones, et al (J. Chem. Soc., pp. 1997-1999 (1938)). Alternatively, 4-phenylbutyric acid can be prepared using thianapthene-2-acetic acid and thianapthene-3-acetic acid as described by Blicke, F. F, et al. (J. Am. Chem. Soc., 70, pp. 3768-3769 (1948)). Phenylbutyric acid can also be synthesized via a Grignard reaction, using benzyl magnesium chloride, albeit in an isolated yield of 16.1% (Gresham, E. L., et al., J. Am. Chem. Soc., 71, p. 2807-2808 (1949)).

4-Phenylbutyric acid and appropriate pharmaceutical salts thereof can also be synthesized using the method of Burzynski, et al. described in U.S. Pat. No. 6,372,938. According to this patent, aromatic compounds are reacted with butyrolactone, followed by neutralization with base. The reaction is reported to be able to be conducted in the presence or absence of a catalyst, although the use of Lewis acid catalysts such as AlCl₃, BF₃, ZnCl₂, etc. is preferred. 4-Phenylbutyric acid is reported to be isolated in approximately 94% yield.

4-Phenylbutyric acid and salts can also be obtained from a variety of commercial sources. For example, sodium phenylbutyrate can be obtained from Triple Crown America, Inc. (Perkasie, Pa., USA) as triButyrate®, as Buphenyl® from Pharmaceutics International, Inc. (Hunt Valley, Calif., USA) or Ucycld Pharma Inc. (Hunt Valley, Calif.), or as AMMONAPS™ from PackPharm Ltd. (Norwich, Norfolk, UK).

(i) Pharmaceutically Acceptable Salts

The therapeutic compound(s) contained within the pharmaceutical formulation can be used in the form of pharmaceutically acceptable salts derived from inorganic or organic acids. For a general review, see P. H. Stahl, et al. “Handbook of Pharmaceutical Salts: Properties, Selection, and Use” (Wiley VCH, Zürich, Switzerland: 2002).

4-Phenylbutyric acid salt suitable for use in the present invention can be an alkali metal salt, an ammonium salt, a substituted ammonium salt, or an acid addition salt. Preferably, the acid salt is an alkali metal salt, wherein “X” in the above general formula is selected from the group of lithium, sodium, potassium, and cesium.

The alkali metal salts can be prepared from the free acid by means well known to those of skill in the art (e.g., reaction of the free carboxylic acid with an alkali metal hydroxide or alkoxide in an appropriate solvent). The acid addition salts of 4-phenylbutyric acid suitable can be generated from the free-base forms of the compounds by reaction of the latter with one equivalent of a suitable, non-toxic, pharmaceutically-acceptable acid, followed by evaporation of the solvent employed for the reaction and recrystallization of the salt, as required. Suitable acids for forming acid addition salts of the compounds used in the controlled-release formulations of the present invention include, but are not limited to, acetic, benzoic, benzenesulfonic, tartaric, hydrobromic, hydrochloric, citric, fumanic, gluconic, glucuronic, glutamic, lactic, malic, maleic, methanesulfonic, palmoic, salicylic, stearic, succinic, sulfuric, and tartaric acids. The class of acids suitable for formation of nontoxic, pharmaceutically-acceptable salts is well known to practitioners of the pharmaceutical formulation arts, and are described, for example in Stahl, P. H., et al., “Handbook of Pharmaceutical Salts”, Wiley-VCH, Weinheim: Germany (2002). The free base of 4-phenylbutyric acid, if required, can be recovered from an acid addition salt thereof by reaction of the salt with a water solution of the salt with a suitable base such as sodium carbonate, sodium hydroxide, and the like.

The salts can be prepared in situ during the final isolation and purification of the compounds of the present invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the present invention include phosphate, tris and acetate.

Pharmaceutically acceptable salts may be also obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids can also be made.

C. Controlled Release Formulation

Controlled release provides many advantages, including reduced fluctuations in drug plasma levels, a reduction in total dose, minimal side-effects, reduced cost of therapy and a high degree of patient compliance. The present invention involves a pharmaceutical formulation having a controlled release, delayed release, or combined delayed and controlled release profile. Alternatively, the formulation may present a combination of an immediate release formulation and a controlled release formulation.

The controlled-release of 4-phenylbutyric acid can be controlled in any way suitable for achieving the desired result. Books describing methods of controlled delivery that are appropriate for the delivery of 4-PBA include: Robert S. Langer, Donald L. Wise, editors; Medical applications of controlled release (Volumes 1 and 2); Boca Raton, Fla.: CRC Press, 1984; and William J. M. Hrushesky, Robert Langer and Felix Theeuwes, editors; Temporal control of drug delivery (series); New York: New York Academy of Sciences, 1991.

A convenient classification of controlled-release systems is based on the mechanism that controls the release of the substance in question. Representative, non-limiting systems encompassed by the present invention include diffusion-controlled, solvent-controlled and chemically-controlled systems. These categories are not absolute, i.e., certain controlled-release formulations may contain aspects of more than one system. For example, in a particular embodiment of the invention, the controlled-release system contains 4-PBA dispersed in a monolithic polymer matrix, wherein release of the drug from the polymer matrix occurs either by diffusion of the drug from the polymer matrix, or by the erosion of the polymer (due to degradation) or by a combination of the two mechanisms.

(i) Diffusion Controlled Systems

In one embodiment of the invention, controlled-release of 4-PBA is achieved using a diffusion-controlled system. Two basic types of diffusion-controlled systems are suitable for use in the present invention. The first type is a reservoir device, in which the drug is enclosed within device in such a way that the rate of drug release is controlled by its permeation through the diffusion barrier. Representative, non-limiting examples of reservoir systems include membranes, capsules, microcapsules, liposomes, and hollow fibers.

The second type of diffusion-controlled system suitable for use in the present invention is a monolithic (matrix) device in which the active agent is dispersed or dissolved in an rate-controlling matrix (e.g., a polymer matrix). 4-PBA is homogeneously dispersed throughout a rate-controlling matrix and the rate of drug release is controlled by diffusion through the matrix.

Release of 4-BPA from both types of diffusion-controlled system is governed by Fick's law of diffusion in which the flux of diffusion (JD) is related to the negative concentration gradient of the drug molecule times the diffusivity of the drug molecule (D). In a reservoir system, the diffusion barrier is essentially uniform and of a non-changing thickness, so that the diffusion rate of 4-PBA can be kept fairly stable throughout the lifetime of the delivery system. In the monolithic matrix type system, the concentration gradient is time dependent and decreases progressively in response to the growing thickness of diffusional path as time increases.

(a) Reservoir Devices

In one embodiment of the present invention, controlled-release of 4-PBA is achieved using a reservoir device. In this design, a reservoir (e.g., solid drug, dilute solution, or highly concentrated drug solution) is surrounded by a diffusion barrier formed at least in part by a rate-controlling material. The only structure effectively limiting the release of 4-PBA in this embodiment is the diffusion barrier surrounding the reservoir. The reservoir system dosage forms may be large, as in the case of a tablet containing a single large reservoir, or multiparticulate, as in the case of a capsule containing a plurality of reservoir particles, each individually coated with a membrane. The membrane can be non-porous, yet permeable to 4-PBA or it may be porous. Reservoir devices include oral, implantable or transdermal systems, for example.

A variety of materials may be used to fabricate the diffusion barrier surrounding the drug reservoir. The materials suitable for fabricating the diffusion barrier of the device are generally those materials capable of forming walls, with or without pores, through which 4-PBA can pass at a controlled rate of release by the process of diffusion or diffusive flow. Suitable materials for forming the diffusion barrier are naturally occurring or synthetic materials, preferably materials that are biologically compatible with body fluids, tissues or organs, and essentially insoluble in body fluids with which the device will come in contact. Generally, the use of rapidly dissolving materials or materials highly soluble in body fluids is to be avoided since dissolution of the barrier or wall of the device would affect the constancy of 4-PBA release, as well as the capability of the system to remain in place for certain uses for prolonged periods of time.

In one aspect, the invention provides a film coat or membrane covering a pharmaceutical core wherein the core includes 4-PBA and optionally one or more pharmaceutically acceptable excipients. In a particular embodiment of the invention, the film coat or membrane contains one or more polymers. For a review of film coating (with particular reference to polymers and their additives) see Kala H., Dittgen M., Moldenhauer H., Zessin G., On the Pharmaceutical Technology of Film Coating. Pharmazie, 34 (11), 1979. (CBDE Translation.) Polymers suitable for use; in the present invention include, for example, cellulose esters, cellulose ethers, acrylic polymers, or a mixture of polymers. Preferred materials include ethyl cellulose, cellulose acetate and cellulose acetate butyrate. Other polymeric membranes that are biologically compatible and do not adversely affect the drugs can be used.

In a particular embodiment of the invention, the polymeric film coating is an enteric polymeric film coating allows the coated solid to pass intact through the stomach to the small intestine, where the drug is then released for absorption through the intestinal mucosa into the human body where it can exert its pharmacologic effects. Non-limiting examples of enteric polymers include cellulose, vinyl, and acrylic derivatives.

In one embodiment of the present invention, sustained release of 4-PBA is achieved through microencapsulation. As used herein, the term “microcapsule” refers to a small particle (i.e., microparticle) that contains 4-PBA surrounded by a shell or coating. The diameter of the microcapsule of the present invention may range from a few microns to a few millimeters. The term nanocapsule is used to refer to capsules smaller than 1 micron, while the terms microcapsule and macrocapsule are used to refer to capsules between 1 and 1000 microns and capsules greater than 1000 microns, respectively. In a particular embodiment, the microcapsule is not a nanocapsule.

The microencapsulation drug delivery system of the present invention may utilize a variety of protective wall or covering materials, including without limitation, proteins, polysaccharides, starches, waxes, fats, polymers and resins. Polymers may be natural, synthetic or synthetically modified natural polymers. Representative, non-limiting polymers include gelatins, fish collagens, rubber arrabicum, silicon rubber albumen, fibrinogens, casein, haemoglobin, zein, alginate, nylon, nylon-polyethylenimine carragheen, agar-agar, chitosan, arabino-galactan, gelan, cellulose, polyvinylalcohol, polyacroleins, polylactic acid, polyglycolic acid polyamides, polyethyleneglycoles, ethyl Styrolmaleinacidanhydride copolymers, cellulosesulphate-poly(dimethyldiallyl)-ammonium chloride, hydroxy-ethyl methacrylate-methyl methacrylate, chitosan-carboxymethyl-cellulose and alginate-polylysine-alginate.

In a particular embodiment of the present invention, the microcapsulate of the present invention utilizes a substantially water-insoluble polymer in order to extend the release rate of the drug from the microcapsule. Representative, non-limiting water-insoluble polymers include poly(lactide-co-glycolide) (PLGA), poly(lactic acid) (PLA) or poly(glycolic acid) (PGA) which are not soluble in aqueous solutions, and therefore the pharmaceuticals contained therein are only released by simple diffusion through the matrix or following degradation of the polymer, thus resulting in controlled release of the drug contained therein over a period of time such as weeks or even months, rather than rapid and accelerated delivery of the drug. See, e.g., U.S. Pat. No. 6,051,259.

4-PBA can be encapsulated in the form of microparticles using a variety of methods. For a review of microencapsulation technology, see Arshady R., Microspheres and Microcapsules: A Survey of Manufacturing Techniques. 1: Suspension and Crosslinking., Polym. Eng. Sci., 30 (15), 1746-1758, 1989; Arshady R., Microspheres and Microcapsules: A Survey of Manufacturing Techniques. 2: Coacervation., Polym. Eng. Sci., 30 (15), 905-914, 1990; Arshady R., Microspheres and Microcapsules: A Survey of Manufacturing Techniques. 3: Solvent Evaporation., Polym. Eng. Sci., 30 (15), 915-924, 1990). Representative, non-limiting techniques suitable for microencapsulation according to the present invention include complex coacervation, interfacial polymerization, in situ polymerization, polymer-polymer phase separation, desolvation, extrusion and gelation (e.g., thermal, ionic). Several methods to reversibly encapsulate biologically active substances are also suitable for use in the present invention, see e.g., U.S. Pat. No. 4,900,556 by Wheatley et al. entitled “System for Delayed and Pulsed Release of Biologically-Active Substances.”

The microcapsule of the present invention can have many different structures ranging from simple droplets of liquid core material surrounded by a spherical shell, to irregularly-shaped particles containing small droplets of core material dispersed in a continuous polymer shell matrix. Non-limiting microcapsule structures include mononuclear spherical, multinuclear spherical, multinuclear irregular, encapsulated mononuclear capsules and dual-walled microcapsulates, for example. Where no distinct coating and core region can be observed, the analogous terms are microparticles, microspheres, micromatrices and microbeads.

The microcapsules are manufactured with a diameter suitable for the intended route of administration. For example, with a diameter of between 0.5 and 8 microns for intravascular administration, a diameter of 1-100 microns for subcutaneous or intramuscular administration, and a diameter of between 0.5 and 5 nm for oral administration for delivery to the gastrointestinal tract or other lumens. A preferred size for administration to the pulmonary system is an aerodynamic diameter of between one and three microns, with an actual diameter of five microns or more.

In one embodiment of the present invention, controlled release of 4-PBA is achieved using aggregate or non-pareil granules of 4-PBA are coated with PH-sensitive, enteric, or sustained release coatings. These granules are then packaged into a capsule or compressed with additional excipients to form a tablet. The dosage form may be a two-piece hardshell capsules containing coated or delayed-release pellets.

(b) Monolithic Matrix Device

In another embodiment of the present invention, controlled release of 4-PBA is achieved using a monolithic matrix device. 4-PBA is homogeneously dispersed throughout a rate-controlling matrix or network and the rate of drug release is controlled by diffusion through the matrix or network. In a particular embodiment of the invention, the matrix or network is a polymer matrix or network. As the release continues, its rate normally decreases with this type of system, since the active agent has a progressively longer distance to travel and therefore requires a longer diffusion time to release. The release characteristics of a monolithic device are dependent upon the solubility of the drug in the polymer matrix. If the matrix is porous, release depends upon the solubility in the sink solution within the particle's pore network, as well as the tortuosity of the network dependent on whether the drug is dispersed in the polymer or dissolved in the polymer. See Singh P et al. J. Pharm. Sci. (1968) 57 (2): 217-226, 1968.

Following the dispersion or dissolution of the drug in a matrix formulation is the form design, the composite solution, suspension or solid must be given a shape or form. Tablet formation is one way of designing a controlled release form. In one embodiment, the matrix is compressed into a tablet.

A wide array of polymers can be employed as the release rate modifying material in the monolithic matrix system of the present invention. In one embodiment, the system is a purely diffusion-controlled system, which is fundamentally stable in the biological environment and does not change its size either through swelling or degradation. In this embodiment of the present invention, the polymer matrix component allows for 4-BPA to diffuse through the pores or macromolecular structure of the polymer upon introduction of the delivery system into the biological environment without inducing any change in the polymer itself. In another embodiment of the invention, the polymer matrix is incapable of releasing its agent or agents until it is placed in an appropriate biological environment, as described further below under “Solvent Controlled Systems” and “Chemically Controlled Systems.”

In one embodiment of the present invention, the monolithic matrix device contains a single polymer type. The single polymer type may be a homopolymer (i.e., incorporating a single monomeric unit) or may be a co-polymer or terpolymer of two or more of these monomeric units, with the monomeric order being random, alternating, block or graft. The polymer may be linear, branched or cross-linked.

In another embodiment of the present invention, the monolithic matrix device contains two ore more polymer types (i.e., a polymer blend). Physical combinations or mixtures of these polymers, copolymers or terpolymers may also be employed. Representative U.S. patent disclosing polymer blends include U.S. Pat. No. 5,128, 143 (Baichwal et al.) entitled “Sustained Release Excipient and Tablet Formation”; U.S. Pat. No. 4,842,866 (Horder et al) entitled “Slow Release Solid Preparation”; U.S. Pat. No. 5,811,126 (Krishnamurthy) entitled “Controlled Release Matrix for Pharmaceuticals”; U.S. Pat. No. 3,965,256 (Leslie) entitled “Slow Release Pharmaceutical Composition”; and U.S. Pat. No. 4,235,870 (Leslie) entitled “Slow Release Pharmaceutical Compositions.” In a polymer blend, the ratio of the various polymer types to each other may be equal or different. For example, in a polymer blend containing two different polymers, the ratio of the first polymer type to the second polymer type may be, for example 10:1, 9:1, 8:1, 7:1, 6:1, 5:1, 4:1, 3:1,2:1, or 1:1.

Polymers suitable for use in the monolithic matrix device of the present invention include naturally occurring polymers, synthetic polymers and synthetically modified natural polymers. The monolithic matrix device of the present device may also contain polymer derivatives. As used herein, “derivatives” include polymers having substitutions, additions of chemical groups, for example, alkyl, alkylene, hydroxylations, oxidations, and other modifications routinely made by those skilled in the art.

The amount of polymer present by weight of the total weight of the finished formulation in the matrix can range, for example from 1% to 3%, 3 to 5%, 5% to 7%, 7% to 10%, 10% to 15%, 15% to 20%, 21% to 30%, 31% to 40%, 41% to 50%, 51% to 60%, 61% to 70%, 71% to 800/o, 81% to 90%, and 91% to 99%. In a diffusion controlled system, an increase in polymer concentration relative to drug concentration can slow the rate of drug release (see R. Baker, Introduction, in R. Bkader, Ed. Controlled Release of Biologically Active Agents (John Wiley & Sons, New York, N.Y. USA, 1988) pp 1-21). In a particular embodiment of the invention, the amount of polymer present by weight is from about 51% to about 99%. The loading level may range, for example from 0-5% (v/v), 5-10%, 10-15%, 15-20%, 20-25%, or greater than 25%. In general, increase in loading level increases the complexity of the monolithic dispersion.

The viscosity of the polymer can be vary, as measured for 2% aqueous solutions at 20° C. It can range from about 15 to about 150,000 cps. In a particular embodiment, the viscosity of the polymer is from about 15-50 cps; 50-100 cps; 100-500 cps; 500-1000 cps; 1000-5000 cps; 5000-10,0000 cps; 10,000-25,000 cps; 25,000-50,000 cps; 50,000-75,000 cps; 75,000-100,000 cps; 100,000-125,000 cps; or 125,000-150,000 cps. It is generally accepted that an increase in viscosity for a polymer can correspond to an increase in the molecular weight of the polymer, an increase in the branching of the polymer or an increase in the degree of substitution of the polymer.

In one embodiment of the present invention, 4-PBA is delivered in a plastic matrix system. Polymers in a plastic matrix system forming insoluble or skeleton matrices that are chemically inert and have a good drug embedding ability. Any polymeric plastic material suitable for use in the present invention provided it is insoluble or substantially insoluble in water, and includes cellulose derivatives such as cellulose acetates, (cellulose acetate butyrate, cellulose acetate propionate, cellulose acetate phthalate, etc.), methyl, ethyl and propyl celluloses; polycarbonates; polystyrenes; alkylacrylates such as polymethyl methacrylate, polyethyl ethacrylate, polyethylene, polyethylene methacrylate and other lower alkyl acrylates; vinyl acetate/vinyl chloride, methyl acrylate/methylmethacrylate vinyl polymers; polyvinylchloride polyurethanes; polyacrylonitriles; and mixtures, combinations and multipolymers (copolymers, terpolymers, etc.) thereof.

Hydrophobic polymers are also suitable for use in the present invention. Hydrophobic and waxy materials are potentially erodable and control the release of drug through pore diffusion and erosion (See, generally, Lordi, N. G., Sustained Release Dosage Forms, in Laclunan L: Lieberman H A and Kanig J L (eds), The Theory and Practice of Industrial Pharmacy. 3rd ed., Varghese Publishing House, Bombay, pp 430456, 1990). Lipophilic matrices can contain fatty excipients including glycerides (e.g., mono-, di- or triglycerides such as stearin, palnitin, laurin, myristin, hydrogenated castor or cottonseed oils, precirol), fatty acids and alcohols (e.g., stearic, palmitic or lauric acids; stearyl, cetyl or cetostearyl alcohols), fatty acid esters (e.g., monostearates of propylene glycol and of sucrose, sucrose distearate) and waxes (e.g., white wax, cachalot wax). Other hydrophobic materials suitable for use in the present invention include, for example, hydrogenated castor oil (HCO), ethylcellulose and carnauba wax. Further examples of lipophilic materials include glyceryl palmitosterate (PRECIROL ATO 5), glyceryl behenate (COMPRITOL 888 ATO) and Hydrogenated castor oil (CUTINA HR).

The polymer used to form the monolithic matrix device of the present invention may be non-degradable. Non-degradable polymers include, for example, polyacrylates, polymers of ethylene-vinyl acetates other acyl substituted cellulose acetates, poly(meth)acrylic acid, polyamides, polyethylene, polypropylene, non-degradable polyurethanes, polystyrene, polyvinyl chloride, polyvinylphenol, poly(vinyl imidazole), hlorosulphonate polyolefins, polyethylene oxide, blends, and copolymers thereof.

and copolymers and mixtures thereof.

Controlled-release of 4-PBA also can be achieved using a composite device consisting of a polymer/4-PBA matrix coated in a polymer containing no 4-PBA. See, for example, Bodmeier R and Paeratakul O. “Drug Release from Laminated Polymeric Films Prepared from Aqueous Latexes” J. Pharm. Sci. (1990) 79 (1): 32-36; Laghoueg N., Paulet J. L., Taverdet J. L., Vergnaud J. M. “Oral Polymer-Drug Devices with a Core and an Erodible Shell for Constant Drug Delivery. Int. J. Pharm. (1989) 50:133-139, 1989.

Coating polymers suitable for use in the present invention include, for example, ethylcellulose, polyvinyl alcohol, hydroxypropylmethylcellulose, olymethyl-methylacrylate, ethylacrylate, polyethylene, polyvinylacetate, polymethacrylate, styrene/maleic copolymer, cellulose acetate pthahate, dellulose acetate pthahate/PEG blend, microcrystalline cellulose, polydextrose, lactose and shellacs.

(ii) Solvent Activated Systems

In one embodiment, the drug delivery system of the present invention is incapable of releasing 4-PBA until it is placed in an appropriate biological environment, such as a solvent-activated system. Solvent activated systems include (i) swellable controlled-release systems; (ii) osmotic systems (i.e., involving transport of water through a semipermeable membrane).

(a) Swellable Matrix Systems

In one embodiment of the present invention, controlled release is achieved in a swellable controlled-release system. In a swelling-controlled device, 4-PBA is dispersed in a swellable matrix. When the drug delivery device is placed in an aqueous environment, water penetrates into the matrix and swelling begins to take place. Hydrogels are materials which swell when placed in excess water. They are able to swell rapidly and retain large amount of water in their swollen structure. The materials do not dissolve in water and maintain three-dimensional networks. Hydrogels are usually made of hydrophilic polymer molecules which are crosslinked either by chemical bonds or other cohesion forces such as ionic interaction, hydrogen bonding or hydrophobic interaction. Hydrogels are elastic solids in the sense that there exist remembered reference configurations to which the system returns even after being deformed for a very long time.

In a particular embodiment of the invention, the matrix is a hydrophilic polymer matrix. The term “hydrophilic”, as used herein, refers to a composition, substance or material, for example, a polymer, which may generally readily associate with water. The hydrophilic polymers that may be employed in the present invention may have domains of varying type, for example, domains which are more hydrophilic and domains which are more hydrophobic, the overall nature of the hydrophilic polymers is preferably hydrophilic, it being understood that this hydrophilicity may vary across a continuum from relatively more hydrophilic to relatively less hydrophilic.

The swellable polymer matrix system of the present invention is capable of absorbing water or other fluids for the purpose of swelling. Drug is normally incorporated in hydrogel polymer in the glassy (dry state). The polymeric chains network will then swell when introduced in aqueous media. The swelling increases the aqueous solvent content within the formulation as well as the polymer mesh size, enabling the drug to diffuse through the gel and into the external environment. (For a general review, see Pedley D. G., Skelly P. J., Tighe B. J., Hydrogels in Biomedical Applications., Br. Polym. J., 12, 99-110, 1980; Ratner B. D., Hoffman A. S., Synthetic Hydrogels for Biomedical Applications., Ch. 1, in Hydrogels for Medical and Related Applications., ACS Symp. Ser., 31, 1-35, 1976).

Representative, non-limiting, hydrophilic polymers suitable for use in a polymer matrix include cellulose derivatives, non-cellulose polysaccharides, polyethylene oxide, polyvinyl alcohols and acrylic acid co-polymers. Cellulose derivatives include, for example, cellulose ethers such as methylcellulose (MC), hydroxypropyl methylcellulose (HPMC) (high, medium and low molecular weight), hydroxyethyl cellulose (HEC), hydroxypropyl cellulose (HPC). For a general review of cellulose ethers, see Salsa T, Veiga F, Pina M E. Oral controlled-release dosage forms. I. Cellulose ether polymers in hydrophilic matrices. Drug Dev Ind Pharm. (1997) 23:929-938; Alderman D A. “A review of cellulose ethers in hydrophilic matrices for oral controlled-release dosage form, Int. J. Pharm. Tech. & Prod. Mfr. (1984) 5, 1-9. Other suitable cellulose derivatives include carboxymethylcellulose, hydroxomethylcellulose, sodium carboxymethyl cellulose and hemicellulose. Non-limiting examples of non-cellulose polysaccharides include galactomannans, guar gum, carob gum, arabic gum, sterculia gum, agar, and alginates (e.g., potassium alginates, sodium alginates). Representative, non-limiting examples of acrylic acid polymers include carbopols 934P and 974P, EUDRAGIT LD 35; Noveon or polycarbophils.

Other hydrophilic polymers include one or more natural or partially or totally synthetic hydrophilic gums such as gum tragacanth, locust bean gum, karaya gum; proteinaceous substances such as pectin; and other hydrophilic polymers such as carboxypolymethylene, gelatin, casein, zein, bentonite, magnesium aluminum silicate, carbomer, zooglan, polysaccharides, modified starch derivatives such as Amazio 721A (American Maize Products) and Pullulan (Hayashibara Biochemical Laboratories, Inc.).

In one embodiment of the present invention, the polymer matrix includes a single hydrophilic polymer type (i.e., a single homopolymer co-polymer or terpolymer) or a hydrophilic polymer blend (i.e., two or more different hydrophilic polymers). One example of a hydrophilic polymer blend includes a include a mixture of a hydroxypropyl methylcellulose and a hydroxypropyl cellulose. In another embodiment of the present invention, the polymer matrix may include a polymer blend of a hydrophilic polymer in combination with a different polymer type, such as a hydrophobic polymer.

The hydrogels of the present invention can include hydrophilic vinyl and acrylic polymers, polysaccharides such as calcium alginate, and poly(ethylene oxide). For example, poly(2-hydroxyethyl methacrylate), poly(acrylic acid), poly(methacrylic acid), poly(N-vinyl-2-pyrolidinone), poly(vinyl alcohol) and their copolymers with each other and with hydrophobic monomers such as methyl methacrylate, vinyl acetate, and the like. Hydrophilic polyurethanes containing large poly(ethylene oxide) blocks can also be used. Hydrogels can also be formed using interpenetrating networks of polymers, which may be formed by addition or by condensation polymerization, the components of which may comprise hydrophilic and hydrophobic monomers such as those just enumerated.

In another embodiment of the present invention, 4-PBA is delivered by an environmentally sensitive hydrogel. In this type of system, swelling is triggered by a change in the environment surrounding the delivery system. The environmentally sensitive hydrogel may be, for example, a pH sensitive hydrogel (e.g., acidic or basic hydrogel); a thermo-sensitive hydrogel; a light-sensitive hydrogel; a ligand-activated hydrogel; an ionic hydrogel; a glucose-sensitive hydrogel; an electrical hydrogel (e.g., polyelectrolyte hydrogel); an ultrasound irradiation-sensitive hydrogel (e.g., ethylene-vinyl alcohol hydrogel); a magnetic-sensitive hydrogel (e.g., magnetic particles dispersed in aligenate microspheres), a chemical species-sensitive hydrogel (e.g., hydrogels containing electron accepting groups); or an enzyme substrate-sensitive hydrogel (e.g., hydrogels containing immobilized enzymes).

To improve sustained release properties in higher pH environments (e.g., the intestines), it may be advantageous to use polymers which dissolve only at higher pHs, either alone or in combination with hydrophilic polymers. Representative, non-limiting examples of polymers that dissolve at higher pH's include acrylic resins, acrylic latex dispersions, cellulose acetate phthalate, and hydroxypropyl methylcellulose phthalate. Enteric coatings consist of pH sensitive polymers. Typically the polymers are carboxylated and interact (swell) very little with water at low pH, whilst at high pH the polymers ionize causing swelling, or dissolving of the polymer. Coatings can therefore be designed to remain intact in the acidic environment of the stomach (protecting either the drug from this environment or the stomach from the drug), but to dissolve in the more alkaline environment of the intestine.

In one embodiment, the polymer matrix is a hyroxypropylmethlcellulose (HPMC) matrix. (See generally Hogan J E. “Hydroxypropylmethylcelluose sustained release technology, Drug Dev. Ind. Pharm. (1989) 15, 975-999). The HPMC matrix may include an HPMC homopolymer, co-polymer or terpolymer. A single HPMC or a mixture of HPMCs of difference molecular weight and structure may be used. HPMC may be used alone, or in a combination with a second polymer type to form a polymer blend.

In a particular embodiment of the invention, the matrix is a blend of HPMC and one or more additional hydrophilic polymers. In one embodiment, the matrix is a blend of (i) HPMC/methylcellulose; (ii) HPMC/hydroxyethylcellulose; (iii) HPMC/hydroxy-ethylmethylcellulose; (iv) HPMC/carboxymethylcellulose; (v) HPMC/ethylcellulose; (vi) HPMC/hydroxypropylcellulose; and (vii) HPMC/microcrystalline cellulose (colloidal).

In another embodiment, the matrix is a blend of HPMC and one or more non-hydrophilic polymers, such as an HPMC/hydrophobic polymer blend.

Commercially available hydroxypropylmethylcellulose is available in different chemical structure and composition, with a methoxyl content ranging from approximately 16.5 to 30 weight-% and a hydroxypropoxyl content within the range of 4 to 32 weight-%, and each of which is available in various viscosity grades. Commercial designations of HPMC represent number average molecular weights ranging from below 10,000 to over 150,000, as calculated from the data in “Handbook of Methocel Cellulose Ether Products” (The Dow Chemical Co., 1974). In one embodiment, the polymer is HPMC of the 2208 USP XXII type, having a molecular weigh of approximately 20,000-250,000, preferably 20,000-120,000, and has a preferred viscosity of 100-1500 cps. Especially suitable are Methocel® K types which produce the fastest swelling, for example Methocel® K100M Premium (Prochem Chemical Company), Methocel® K100LV, Methocell®K4M and Methocel® K5M (brand names, Dow Chemical Company) or the virtually equivalent Metolose® 90SH type, for example Metolose® 90SH100, Metolose® 90SH4,000 and Metolose® 90SH15,000 (brand names, Shin-Etsu Chemical Co. Ltd). In one embodiment, approximately 5-50% by weight, preferably 10-40% by weight, HMPC are used, based in the final weight of the tablet or capsule filling. In one embodiment, the polymer is HPMC and the viscosity ranges from about 5 to 100,000 cps (mPa.sec). In a particular embodiment, the HPMC polymer is either HPMC. K100M (having a viscosity of 100,000 cps) or HMPC K15M (having a viscosity of 15,000 cps).

As the viscosity of the polymer is increased, the rate of release of the phenylbutyrate is increased. Surprisingly, as the viscosity of the HPMC is increased from 15,000 to 100,000, the rate of delivery of the phenylbutyrate and the overall amount of phenylbutyrate delivered increases. This behavior is quite unexpected as it is generally expected in the art that the rate of delivery of components in a controlled release matrix will decrease as the viscosity of the release rate modifier increase (Wan L S C, Heng P W S, Wong L F. “Relationship between polymer viscosity and drug release from a matrix system” Pharm. Res. (1992) 9, 1510-1514. s. Accordingly, one embodiment of the invention provides a controlled release formulation wherein the polymer is present in an amount sufficient to render the release rate of the drug dependent upon the viscosity of the polymer.

(b) Osmotically Controlled System

Osmotically controlled systems are also suitable for use in the present invention. In this embodiment, an osmotic pressure gradient is created to draw an aqueous fluid into a compartment containing 4-PBA, causing 4-PBA to be delivered. Osmotic delivery systems include a compartment containing 4-PBA and an osmotic agent which draws an aqueous fluid through the walls of the compartment, causing swelling of the osmotic agent and delivery of 4-PBA.

The osmotic delivery system of the present invention may include a single compartment containing both the beneficial agent and the osmotic agent. This device releases 4-PBA by allowing fluid to be imbibed through the wall of the compartment at a rate determined by the permeability of the wall and the osmotic pressure gradient across the wall. The fluid imbibed into the device mixes with the therapeutic agent to form an aqueous solution which is dispensed through an exit passageway of the device. See, e.g., U.S. Pat. Nos. 3,845,770 and 3,916,899.

In another embodiment, there is more than one compartment. For example, there is a first therapeutic agent compartment separated by a film or piston from a second osmotic compartment. In this embodiment, 4-PBA is delivered by imbibing fluid through the wall of the device into the osmotic compartment. As the osmotic compartment fills with fluid, the osmotic agent within the compartment swells and acts as a driving force causing the film or piston to move against the 4-PBA and deliver 4-PBA. See, e.g., U.S. Pat. Nos. 4,111,202; 4,111,203; and 4,203,439. See also U.S. Pat. Nos. 5,728,396; 6,464,688; 6,632,217; 6,840,931

4-BPA can be delivered at a controlled rate which can vary depending on many factors including the osmotic material used, the permeability of the walls, and the physical configuration of the delivery device. The wall may be, for example, a semi-permeable membrane. The osmotic agent may be an osmagent, an osmopolymer, or a mixture of the two. An osmagent is a non-volatile species which is soluble in water and create the osmotic radiant driving the osmotic inflow of water, vary widely. Examples are well known in the art and include magnesium sulfate, magnesium chloride, potassium sulfate, sodium chloride, sodium sulfate, lithium sulfate, sodium phosphate, potassium phosphate, d-mannitol, sorbitol, inositol, urea, magnesium succinate, tartaric acid, raffinose, and various monosaccharides, oligosaccharides and polysaccharides such as sucrose, glucose, lactose, fructose, and dextran, as well as mixtures of any of these various species. Osmopolymers may be of plant or animal origin, or synthetic, and examples of osmopolymers are well known in the art. Examples include: poly(hydroxy-alkyl methacrylates) with molecular weight of 30,000 to 5,000,000, poly(vinylpyrrolidone) with molecular weight of 10,000 to 360,000, anionic and cationic hydrogels, polyelectrolyte complexes, poly(vinyl alcohol) having low acetate residual, optionally cross-linked with glyoxal, formaldehyde or glutaraldehyde and having a degree of polymerization of 200 to 30,000, a mixture of methyl cellulose, cross-linked agar and carboxymethylcellulose, a mixture of hydroxypropl methycellulose and sodium carboxymethylcellulose, polymers of N-vinyllactams, polyoxyethylene-polyoxypropylene gels, polyoxybutylene-polyethylene block copolymer gels, carob gum, polyacrylic gels, polyester gels, polyurea gels, polyether gels, polyamide gels, polypeptide gels, polyamino acid gels, polycellulosic gels, carbopol acidic carboxy polymers having molecular weights of 250,000 to 4,000,000, Cyanamer polyacrylamides, cross-linked indene-maleic anhydride polymers, Good-Rite polyacrylic acids having molecular weights of 80,000 to 200,000, Polyox Polyethylene oxide polymers having molecular weights of 100,000 to 5,000,000, starch graft copolymers, and Aqua-Keeps acrylate polymer polysaccharides.

(iii) Chemical Controlled Systems

In one embodiment of the present invention, the drug delivery system is a chemically controlled system. Chemical control can be achieved, for example, using bioerodable polymers or pendant chains. achieved using bioerodible polymers or pendant chains. One advantage of a biodegradable systems is that the bioerodible devices are eventually absorbed by the body and thus need not be removed surgically. In a pendant chain system, the drug is covalently bound to the polymer and is released by bond scission owing to water or enzymes. In solvent-activated controlled systems, the active agent is dissolved or dispersed within a polymeric matrix and is not able to diffuse through that matrix.

(a) Erosion Based System

In one embodiment of the present invention, controlled release of 4-PBA is achieved using a biodegradable monolithic polymer matrix. In this type of system, the bioactive agent is ideally distributed uniformly throughout a polymer in the same way as in monolithic systems.

Biologically degradable polymers are polymers which degrade to smaller fragments due to chemicals present inside the body. In generally, biologically degradable polymers are either (i) biodegradable polymers or (ii) bioabsorbable polymers. Biodegradable polymers degrade to smaller fragments by enzymes, whereas bioabsorbable polymers degrade in the presence of other chemicals in the body.

As with the diffusion method, the drug is contained within a biologically degradable polymer membrane or matrix. As the polymer degrades, the drug is released into the body. Degradation of polymers occurs via three major mechanisms. See generally, Langer, R., Accounts of Chemical Research, 1993, 26, 537-542. In the first mechanism, water-soluble polymers are made insoluble by cross-linking them together. When the cross-links are broken at some point in the body, the polymer will dissolve. In the second mechanism, water-insoluble polymers are made soluble by hydrolysis or ionization of side groups. In the third mechanism, insoluble polymers are cleaved into soluble monomers. These mechanisms can be used alone, or in combination. The erosion process occurs either in bulk (wherein the matrix degrades uniformly) or at the polymer's surface (whereby release rates are related to the polymer's surface area).

Biodegradable polymers include (i) naturally occurring polymers; (ii) modified natural polymers (i.e., chemically or enzymatically modified polymers; and (iii) synthetic polymers. Representative, non-limiting, naturally occurring biodegradable polymers include alginate, dextrin, cellulose, collagen, chitosan and proteins such as albumin, zein and copolymers and blends thereof, alone or in combination with synthetic polymers. In general, these materials degrade either by enzymatic hydrolysis or exposure to water in vivo, by surface or bulk erosion.

Natural occurring polymers can be modified, for example, by substitutions, additions of chemical groups, for example, alkyl, alkylene, hydroxylations, oxidations, and other modifications routinely made by those skilled in the art. Non-limiting examples of chemical modification of natural polymers include, for example, crosslinking of gelatin using formaldehyde, crosslinking of chitosan using glutaraldehyde and chemical modification of cellulose to give cellulose acetate. Representative enzymatic modification of naturally occurring polymers include the modification of lignin using horseradish peroxidase and modification of chitosan using tyrosine.

Synthetic biodegradable polymers include, for example polyanhydrides, polyesters, polyacrylic acids polyurethanes, polyphosphoesters and polyphosphazenes and poly(methyl methacrylates. Preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof. Other synthetic biodegradable polymers include poly(ethylene terephthalate), poly(butic acid), poly(valeric acid), poly(lactide-co-caprolactone), polyanhydrides, polyorthoesters and blends and copolymers thereof.

Several polymers which degrade into naturally occurring materials have also been described, such as crosslinking gelatin, hyaluronic acid (della Valle et al. U.S. Pat. No. 4,987,744 and U.S. Pat. No. 4,957,744) and polyaminoacids (Miyake et al., 1974), which spurred the usage of polyesters by Holland et al. Controlled Release, 4,155,1986 and alph-hydroxy acids (i.e. lactic acid and glycolic acid), which remain the most widely used biodegradable materials for applications ranging from closure devices (sutures and staples) to drug delivery systems (Smith et al. U.S. Pat. No. 4,741,337; Spilizeqski et al. J. Control. Rel., 2, 197, 1985). Polymers used in surface degradation systems are usually highly hydrophobic yet contain water-labile linkages. See Langer, R., Science (1990): 249, 1527-1533.

Other biodegradable polymers useful in the present invention include starch-polyester alloys; styrene-maleic anhydride copolymers; poly(methylvinyl ether-maleic acid); starch; starch-PCL blends; polylactic acid (PLA)-starch blends; polylactic acid; poly(lactic acid-glycolic acid) copolymers; polylactide, polyglycolide; polyactide co-glycolide PCL; cellulose esters; cellulose acetate butyrate; starch esters; starch ester-aliphatic polyester blends; modified corn starch; polycaprolactone; poly(n-amylmethacrylate); ethyl cellulose; wood rosin; polyvinylalcohol (PVOH); polyhydroxybutyrate-valerate (PHBV); biodegradable aliphatic polyesters; and polyhydroxybutyrate (PHB) and polyhydroxy acids

Degradation of lactide based polymers and in general all hydrolytically degradable polymers, depends on: (i) chemical composition; (ii) crystallinity; and (iii) hydrophilicity. In general, the rate of degradation of polymers depends the type of degradable bonds present on the polymer (e.g., anhydride>esters>amides). The higher the cystallinity, the slower the rate of degradation. Among the polylactides, DL-PLA degrades faster than L-PLA, as a result of lesser crystallinity. The most hydrophobic the polymer, the slower the rate of degradation. Polyactides, for example, are more hydrophobic than PLGA, and degrade more slowly. The hydrophobic end-capped PLGA polymers degrade faster than the carboxyl-ended PLGA.) Other general principles covering release rates of biodegradable polymers include the following: (i) polymers with heteroatoms in backbone>polymers with C-C backbones; (ii) higher molecular weight polymers degrade more slowly than lower molecular weight polymers; and (iii) synthetic step-growth or condensation polymers are generally biodegradable.

In another embodiment, controlled-release of 4-PBA is achieved using a pore-forming wax. In this type of system, 4-PBA is incorporated into a wax base (e.g., paraffin) via tabletting. The rate of release of 4-PBA would be dependent upon the rate of erosion. In this embodiment, 4-PBA and a water soluble excipient (e.g., polymer or salt) are introduced into a wax or wax-like compound and then placed in an aqueous environment in order to allow the water soluble polymer to dissolve out of the wax, resulting in the formation of pores. Upon contact with the gastrointestinal fluid, the pores facilitate erosion of the wax and the subsequent release of 4-PBA.

In another embodiment, controlled-release of 4-PBA is achieved using a pendant device. See, e.g., Chafi N., Montheard J. P., Vergnaud J. M. “Release of 2-Aminothiazole from Polymeric Carriers” Int. J. Pharm., (1992): 67 (3):265-274; Chafi N., Kolli M., Vergnaud J. M., Montheard J. P., “Amines Release from Schiff Bases Polymers and Diffusion from Dosage Forms with Eudragit R L in Acidic Medium” J. Appl. Polym. Sci., (1991) 43 (10):1837-1847). In a pendant chain system, 4-PBA is covalently bound to the polymer and is released by bond scission owing to water or enzymes. Chemical bonding of 4-PBA to polymers can be accomplished in several general ways based on methods of synthesis: (a) reaction on preformed polymers, (b) reactions on naturally-occurring polymers, (c) polymerization of vinyl monomers containing the active ingredient, and (d) step growth polymerizations. See also Scholsky K. M., Fitch R. M., Controlled Release of Pendant Bioactive Materials from Acrylic Polymer Colloids., J. Controlled Release (1986) 3: 87-108;

(iv) Geometrical-Physical Systems

In another embodiment, geometrical-physical systems are used to provide controlled-release 4-PBA. This type of system incorporates 4-PBA into a layer a layer or core, which is then formed into a pellet and altered by physical means to effect and control the rate or erosion or dissolution of the dosage form. Surface-area modifications are used to retard the burst release or increase the extent of the release of 4-PBA from tablet cores that possess diffusion limitations. The physically-altered pellet may then be incorporated alone or in combination with other modified pellets and excipients into a capsule or tablet. Representative geometrical-physical systems include enteric-coated tablet, modified-core tablet systems (e.g., Procise®, GlaxoSmithKline; Smartrix®, Smartrix Technologies).

(v) Other Techniques for Achieving Sustained Release

Other controlled-release strategies suitable for delivery of 4-PBA may include, for example, electrically stimulated release devices (e.g., Yuk S. H., Cho S. H., Lee H. B. “Electric Current-Sensitive Drug Delivery Systems Using Sodium Alginate/Polyacrylic Acid Composites” Pharm. Res. (1992) 9 (7): 955-957); microballoons and microsponges (Kawashima Y., Niwa T., Takeuchi H., Hino T., Itoh Y., “Hollow Macrospheres for use as a Floating Controlled Drug Delivery System in the Stomach” J. Pharm. Sci. (1992) 82 (2): 135-140; Kawashima Y., Niwa T., Takeuchi H., Hino T., Itoh Y., “Control of Prolonged Drug Release and Compression Properties of Ibuprofen Microsponges with Acrylic Polymer, Eudragit RS, by changing their Intraparticle Density” Chem. Pharm. Bull. (1992) 40 (1): 196-201).

Ion-exchanged/complex formation systems are also contemplated to achieve sustain release of 4-PBA according to the present invention. These ion exchange resin-drug or complex-drug systems deliver drugs through ion exchange in stomach or intestines via pH controlled release. Gradient matrix systems are also suitable for use in the present invention. In this type of system, hydrogels can provide zero order kinetics through a gradient concentration across their spherical membrane. Multi-layered tablets can also be used in the present invention.

Non-limiting examples of U.S. patents that describe controlled release formulations are: U.S. Pat. No. 5,356,630 to Laurencin et al. (Delivery System for Controlled Release of Bioactive Factors); U.S. Pat. No. 5,797,898 to Santini, Jr. et al. (Microchip Drug Delivery Devices); U.S. Pat. No. 5,874,064 to Edwards et al. (Aerodynamically Light Particles for Pulmonary Drug Delivery); U.S. Pat. No. 5,548,035 to Kim et al. (Biodegradable Copolymer as Drug Delivery Matrix Comprising Polyethyleneoxide and Aliphatic Polyester Blocks); U.S. Pat. No. 5,532,287 to Savage et al. (Radiation Cured Drug Release Controlling Membrane); U.S. Pat. No. 5,284,831 to Kahl et al. (Drug Delivery Porphyrin Composition and Methods); U.S. Pat. No. 5,741,329 to Agrawal et al. (Methods of Controlling the pH in the Vicinity of Biodegradable Implants); U.S. Pat. No. 5,820,883 to Tice et al. (Methods for Delivering Bioactive Agents into and Through the Mucosally-Associated Lymphoid Tissues and Controlling Their Release);U.S. Pat. No. 5,955,068 to Gouin et al. (Biodegradable polyanhydrides Derived from Dimers of Bile Acids and Use Thereof as Controlled Drug Release Systems); U.S. Pat. No. 6,001,395 to Coombes et al. (Polymeric Lamellar Substrate Particles for Drug Delivery); U.S. Pat. No. 6,013,853 to Athanasiou et al. (Continuous Release Polymeric Implant Carriers); U.S. Pat. No. 6,060,582 to Hubbell et al. (Photopolymerizable Biodegradable Hydrogels as Tissue Contacting Materials and Controlled Release Carriers); U.S. Pat. No. 6,113,943 to Okada et al. (Sustained-Release Preparation Capable of Releasing a Physiologically Active Substance); and PCT Publication No. WO 99/59548 to Oh et al. (Controlled Drug Delivery System Using the Conjugation of Drug to Biodegradable Polyester); U.S. Pat. No. 6,123,861 (Fabrication of Microchip Drug Delivery Devices); U.S. Pat. No. 6,060,082 (Polymerized Liposomes Targeted to M cells and Useful for Oral or Mucosal Drug Delivery); U.S. Pat. No. 6,041,253 (Effect of Electric Field and Ultrasound for Transdermal Drug Delivery); U.S. Pat. No. 6,018,678 (Transdermal protein delivery or measurement using low-frequency sonophoresis); U.S. Pat. No. 6,007,845 Nanoparticles And Microparticles Of Non-Linear Hydrophilic-Hydrophobic Multiblock Copolymers; U.S. Pat. No. 6,004,534 Targeted Polymerized Liposomes For Improved Drug Delivery; U.S. Pat. No. 6,002,961 Transdermal Protein Delivery Using Low-Frequency Sonophoresis; U.S. Pat. No. 5,985,309 Preparation Of Particles For Inhalation; U.S. Pat. No. 5,947,921 Chemical And Physical Enhancers And Ultrasound For Transdermal Drug Delivery; U.S. Pat. No. 5,912,017 Multiwall Polymeric Microspheres; U.S. Pat. No. 5,911,223 Introduction Of Modifying Agents Into Skin By Electroporation; U.S. Pat. No. 5,874,064 Aerodynamically Light Particles For Pulmonary Drug Delivery; U.S. Pat. No. 5,855,913 Particles Incorporating Surfactants For Pulmonary Drug Delivery; U.S. Pat. No. 5,846,565 Controlled Local Delivery Of Chemotherapeutic Agents For Treating Solid Tumors; U.S. Pat. No. 5,837,752 Semi-Interpenetrating Polymer Networks; U.S. Pat. No. 5,814,599 Transdermal Delivery Of Encapsulated Drugs; U.S. Pat. No. 5,804,178 Implantation Of Cell-Matrix Structure Adjacent Mesentery, Omentum Or Peritoneum Tissue; U.S. Pat. No. 5,797,898 Microchip Drug Delivery Devices; U.S. Pat. No. 5,770,417 Three-Dimensional Fibrous Scaffold Containing Attached Cells For Producing Vascularized Tissue In vivo; U.S. Pat. No. 5,770,193 Preparation Of Three-Dimensional Fibrous Scaffold For Attaching Cells To Produce Vascularized Tissue In vivo; U.S. Pat. No. 5,762,904 Oral Delivery Of Vaccines Using Polymerized Liposomes; U.S. Pat. No. 5,759,830 Three-Dimensional Fibrous Scaffold Containing Attached Cells For Producing Vascularized Tissue In vivo; U.S. Pat. No. 5,749,847 Delivery Of Nucleotides Into Organisms By Electroporation; U.S. Pat. No. 5,736,372 Biodegradable Synthetic Polymeric Fibrous Matrix Containing Chondrocyte For In vivo Production Of A Cartilaginous Structure; U.S. Pat. No. 5,718,921 Microspheres Comprising Polymer And Drug Dispersed There Within; U.S. Pat. No. 5,696,175 Preparation Of Bonded Fiber Structures For Cell Implantation; U.S. Pat. No. 5,667,491 Method For Rapid Temporal Control Of Molecular Transport Across Tissue; U.S. Pat. No. 5,654,381 Functionalized Polyester Graft Copolymers; U.S. Pat. No. 5,651,986 Controlled Local Delivery Of Chemotherapeutic Agents For Treating Solid Tumors; U.S. Pat. No. 5,629,009 Delivery System For Controlled Release Of Bioactive Factors; U.S. Pat. No. 5,626,862 Controlled Local Delivery Of Chemotherapeutic Agents For Treating Solid Tumors; U.S. Pat. No. 5,593,974 Localized Oligonucleotide Therapy; U.S. Pat. No. 5,578,325 Nanoparticles And Microparticles Of Non-Linear Hydrophilic-Hydrophobic Multiblock Copolymers; U.S. Pat. No. 5,562,099 Polymeric Microparticles Containing Agents For Imaging; U.S. Pat. No. 5,545,409 Delivery System For Controlled Release Of Bioactive Factors; U.S. Pat. No. 5,543,158 Biodegradable Injectable Nanoparticles; U.S. Pat. No. 5,514,378 Biocompatible Polymer Membranes And Methods Of Preparation Of Three Dimensional Membrane Structures; U.S. Pat. No. 5,512,600 Preparation Of Bonded Fiber Structures For Cell Implantation; U.S. Pat. No. 5,500,161 Method For Making Hydrophobic Polymeric Microparticles; U.S. Pat. No. 5,487,390 Gas-filled polymeric microbubbles for ultrasound imaging; U.S. Pat. No. 5,399,665 Biodegradable polymers for cell transplantation; U.S. Pat. No. 5,356,630 Delivery system for controlled release of bioactive factors; U.S. Pat. No. 5,330,768 Controlled drug delivery using polymer/pluronic blends; U.S. Pat. No. 5,286,763 Bioerodible polymers for drug delivery in bone; U.S. Pat. No. 5,149,543 Ionically cross-linked polymeric microcapsules; U.S. Pat. No. 5,128,420 Method of making hydroxamic acid polymers from primary amide polymers; U.S. Pat. No. 5,122,367 Polyanhydride bioerodible controlled release implants for administration of stabilized growth hormone; U.S. Pat. No. 5,100,668 Controlled release systems containing heparin and growth factors; U.S. Pat. No. 5,019,379 Unsaturated polyanhydrides; U.S. Pat. No. 5,010,167 Poly(amide-and imide-co-anhydride) for biological application; S. Patent No. 4,948,587 Ultrasound enhancement of transbuccal drug delivery; U.S. Pat. No. 4,946,929 Bioerodible articles useful as implants and prostheses having predictable degradation rates; U.S. Pat. No. 4,933,431 One step preparation of poly(amide-anhydride); U.S. Pat. No. 4,933,185 System for controlled release of biologically active compounds; U.S. Pat. No. 4,921,757 System for delayed and pulsed release of biologically active substances; U.S. Pat. No. 4,916,204 Pure polyanhydride from dicarboxylic acid and coupling agent; U.S. Pat. No. 4,906,474 Bioerodible polyanhydrides for controlled drug delivery; U.S. Pat. No. 4,900,556 System for delayed and pulsed release of biologically active substances; U.S. Pat. No. 4,898,734 Polymer composite for controlled release or membrane formation; U.S. Pat. No. 4,891,225 Bioerodible polyanhydrides for controlled drug delivery; U.S. Pat. No. 4,888,176 Controlled drug delivery high molecular weight polyanhydrides; U.S. Pat. No. 4,886,870 Bioerodible articles useful as implants and prostheses having predictable degradation rates; U.S. Pat. No. 4,863,735 Biodegradable polymeric drug delivery system with adjuvant activity; U.S. Pat. No. 4,863,611 Extracorporeal reactors containing immobilized species; U.S. Pat. No. 4,861,627 Preparation of multiwall polymeric microcapsules; U.S. Pat. No. 4,857,311 Polyanhydrides with improved hydrolytic degradation properties; U.S. Pat. No. 4,846,786 Bioreactor containing suspended, immobilized species; U.S. Pat. No. 4,806,621 Biocompatible, bioerodible, hydrophobic, implantable polyimino carbonate article; U.S. Pat. No. 4,789,724 Preparation of anhydride copolymers; U.S. Pat. No. 4,780,212 Ultrasound enhancement of membrane permeability; U.S. Pat. No. 4,779,806 Ultrasonically modulated polymeric devices for delivering compositions; U.S. Pat. No. 4,767,402 Ultrasound enhancement of transdermal drug delivery; U.S. Pat. No. 4,757,128 High molecular weight polyanhydride and preparation thereof; U.S. Pat. No. 4,657,543 Ultrasonically modulated polymeric devices for delivering compositions; U.S. Pat. No. 4,638,045 Non-peptide polyamino acid bioerodible polymers; U.S. Pat. No. 4,591,496 Process for making systems for the controlled release of macromolecules.

Non-limiting examples of other polymers suitable for use in the controlled-release drug delivery system according to the present invention include gelatins, fish collagens, rubber arrabicum, silicon rubber albumen, fibrinogens, casein, haemoglobin, zein, alginate, nylon, nylon-polyethylenimine carragheen, agar-agar, chitosan, arabino-galactan, gelan, cellulose, polyvinylalcohol, polyacroleins, polylactic acid, polyglycolic acid polyamides, polyethyleneglycoles, ethyl Styrolmaleinacidanhydride copolymers, cellulosesulphate-poly(dimethyldiallyl)-ammonium chloride, hydroxy-ethyl methacrylate-methyl methacrylate, chitosan-carboxymethyl-cellulose, alginate-polylysine-alginate, cellulose ester, cellulose ether, an acrylic polymer, ethyl cellulose, cellulose acetate, cellulose acetate butyrate, poly(lactide-co-glycolide) (PLGA), poly(lactic acid) (PLA), poly(glycolic acid) (PGA), polyvinyl chloride, polyethylene, vinyl acetate/vinyl chloride copolymers, polymethylmethacrylates, polyamides, silicones, polystyrene low density polyethylene, ethylene-vinylacetate copolymers, styrene-butadiene-styrene copolymers, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), hydro glycerides (e.g., mono-, di- or triglycerides such as stearin, palnitin, laurin, myristin, hydrogenated castor or cottonseed oils, precirol), fatty acids and alcohols (e.g., stearic, palmitic or lauric acids; stearyl, cetyl or cetostearyl alcohols), fatty acid esters (e.g., monostearates of propylene glycol and of sucrose, sucrose distearate), waxes (e.g., white wax, cachalot wax), hydrogenated castor oil (HCO), ethylcellulose, poly(hydroxy acids), poly(lactic acid), poly(glycolic acid), poly(lactic acid-co-glycolic acid), poly(lactide), poly(glycolide), poly(lactide-co-glycolide), polyanhydrides, polyorthoesters, polyamides, polycarbonates, polyalkylenes, polyethylene and polypropylene, polyalkylene glycols, poly(ethylene glycol), polyalkylene oxides, poly(ethylene oxide), polyalkylene terepthalates, poly(ethylene terephthalate), polyvinyl alcohols, polyvinyl ethers, polyvinyl esters, poly (dimethyl silicone) polymethacrylate, polymethylmethacrylate, polyvinyl halides, poly(vinyl chloride), polyvinylpyrrolidone, polysiloxanes, poly(vinyl alcohols), poly(vinyl acetate), poly (ethylene/vinyl acetate) polystyrene, polyurethanes, derivativized celluloses, alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, polymers of acrylic acid, methacrylic acid, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(butyric acid), poly(valeric acid), poly(lactide-co-caprolactone), polyphosphazenes, poly(vinyl alcohols), polyamides, polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, polyacrylates, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate), albumin, prolamines, ellulose, dextrans, polyhylauronic acid, polyhydroxyalkanoates, polyhydroxybutyrate, lkyl celluloses, hydroxyalkyl celluloses, nitrocelluloses, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, ellulose sulfate sodium salt, ethylcellulose, polyvinyl alcohol, hydroxypropylmethylcellulose, olymethylmethylacrylate, ethylacrylate, polyethylene, polyvinylacetate, polymethacrylate, styrene/maleic copolymer, cellulose acetate pthahate, dellulose acetate pthahate/PEG blend, microcrystalline cellulose, polydextrose, lactose, shellacs, cellulose derivatives, non-cellulose polysaccharides, polyethylene oxide, polyvinyl alcohols, acrylic acid copolymers methylcellulose, hydroxypropyl methylcellulose (HPMC) (high, medium and low molecular weight), hydroxyethyl cellulose, hydroxypropyl cellulose, carboxymethylcellulose, hydroxomethylcellulose, hemicellulose, methylcellulose, galactomannans, guar gum, carob gum, gum arabic, sterculia gum, agar, alginates, carbopols 934P and 974P, polyvinyl alcohol (PVA)/polyvinyl pyrrolidone (PVP), gum tragacanth, locust bean gum, karaya gum, proteinaceous substances (e.g., pectin, carragee) carboxypolymethylene, gelatin, bentonite, magnesium aluminum silicate, carbomer, zooglan, polysaccharides, modified starch derivatives (e.g., Amazio 721A), hydrophilic viny, acrylic polymers, poly(2-hydroxyethyl methacrylate), poly(acrylic acid), poly(methacrylic acid), poly(N-vinyl-2-pyrolidinone), poly(vinyl alcohol) polyanhydrides, polyesters, polyacrylic acids polyurethanes, polyphosphoesters and polyphosphazenes and poly(methyl methacrylates, polyanhydrides, poly(ortho)esters, polyurethanes, poly(butyric acid), poly(valeric acid), poly(lactide-co-caprolactone), poly(ethylene terephthalate), poly(butic acid), poly(valeric acid), poly(lactide-co-caprolactone), polyanhydrides, starch-polyester alloys; styrene-maleic anhydride copolymers, poly(methylvinyl ether-maleic acid), starch, starch-PCL blends, polylactic acid (PLA)-starch blends, polylactic acid, poly(lactic acid-glycolic acid) copolymers, polylactide, polyglycolide; polyactide co-glycolide PC, starch esters, starch ester-aliphatic polyester blends, modified corn starch, polycaprolactone, poly(n-amylmethacrylate), ethyl cellulose, wood rosin, polyvinylalcohol (PVOH), polyhydroxybutyrate-valerate (PHBV), biodegradable aliphatic polyesters, polyhydroxybutyrate (PHB) and polyhydroxy acids.

(vi) Excipients

The controlled-release formulation can also include a number of other excipients and diluents. The term “excipient” refers to substances that are commonly provided within finished dosage forms, and include vehicles, binders, disintegrants, fillers (diluents), lubricants, glidants (flow enhancers), compression aids, colors, flavors sweeteners, preservatives, suspending/dispersing agents, film formers/coatings and printing inks.

Lubricants may include, for example, magnesium stearate, calcium stearate, zinc stearate, powdered stearic acid, hydrogenated vegetable oils, talc, polyethylene glycol, and mineral oil;

Disintegrants include starches such as corn starch, potato starch, pregelatinized and modified starches thereof, cellulosic agents such as Ac-di-sol, montmorrilonite clays, cross-linked PVP, sweeteners, bentonite and VEEGUM™, microcrystalline cellulose, alginates, sodium starch glycolate, gums such as agar, guar, locust bean, karaya, pectin and tragacanth.

The present formulations may contain flavorants or sweetening agents. As used herein, the term “flavorant” is intended to mean a compound used to impart a pleasant flavor and often odor to a pharmaceutical preparation. Flavorants may be natural or synthetic or combinations thereof. These may include, for example, cinnamon oil, oil of wintergreen, peppermint oils, clove oil, bay oil, anise oil, eucalyptus, thyme oil, cedar leave oil, oil of nutmeg, oil of sage, oil of bitter almond, cassia oil, vanilla, citrus oil, including lemon, orange, grape, lime and grapefruit, and fruit essences, including apple, pear, peach, strawberry, raspberry, cherry, plum, pineapple, apricot and so forth. The amount of flavoring may depend on a number of factors, including the organoleptic effect desired. Sweetening agents may include, for example, aspartame, dextrose, glycerin, mannitol, saccharin sodium, sorbitol and sucrose and the like.

The 4-phenylbutyric acid containing pharmaceutical formulation of the present invention may require particular binders in order to obtain a suitable control-release product. Suitable hydrophobic binders include but are not limited to cellulose acetate butyrate, cellulose acetate propionate, cellulose propionate high molecular weight (200,000), cellulose propionate medium molecular weight (75,000), cellulose propionate low molecular weight (25,000), cellulose acetate, cellulose nitrate, ethylcellulose, polyvinyl acetate, and the like. Suitable hydrophilic binders include polyvinylpyrrolidone, vinyl alcohol polymer, polyethylene oxide, water soluble or water swellable cellulose and starch derivatives and others known to those of ordinary skill in the art.

Other binders suitable for use include, for example, acacia, tragacanth, gelatin, starch, cellulose materials such as methyl cellulose and sodium carboxymethyl cellulose, alginic acids and salts thereof, polyethylene glycol, guar gum, polysaccharide, sugars (e.g., lactose, sucrose), invert sugars, poloxomers (PLURONIC™ F68, PLURONIC™ F127), collagen, albumin, gelatin, cellulosics in nonaqueous solvents, pregelatinized starch, starch paste and combinations of the above and the like. Other binders include, for example, polypropylene glycol, polyoxyethylene-polypropylene copolymer, polyethylene ester, polyethylene glycol, polyethylene sorbitan ester, polyethylene oxide or combinations thereof and others known to those of ordinary skill in the art.

The pharmaceutical formulations of the present invention may also contain diluents. The term diluent is intended to mean inert substances used as fillers to create the desired bulk, flow properties, and compression characteristics in the preparation of tablets and capsules. Such compounds include, by way of example and without limitation, dibasic calcium phosphate, kaolin clay, fructose, sucrose, dextrose, lactose, mannitol, microcrystalline cellulose, powdered cellulose, precipitated calcium carbonate, sorbitol, calcium sulfate, starch and the like.

The formulations may contain colorants. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and the like. Coloring agents can also include titanium dioxide, natural coloring agents such as grape skin extract, beet red powder, betacarotene, annato, carmine, turmeric, paprika and the like.

The solid dosage forms of tablets, capsules, pills, and granules can be prepared with coatings, films or shells such as enteric coatings and other coatings well known in the pharmaceutical formulating art. The coating may be of various types, including sugar coating, film coating, or enteric coating. Sugar coating is water-based and results in a thickened covering around a formed tablet. Sugar-coated tablets generally dissolve at the higher pH values of the intestines. A film coat is a thin cover around a formed tablet or bead. Unless it is an enteric coat, the film coat will dissolve in the stomach. An enteric coated tablet or bead will pass through the stomach and break up in the intestines.

The film coating can be a neutral film coating or film coating that delays the release of the active ingredient. A film coating having no retarding action consists, for example, of film-formers, pigments, anti-adhesive agents and plasticizers. These film formers may consist of fast-dissolving constituents. In one embodiment, the film former is low-viscocity hydropropylmethylcellulose type 2910 USP XXII, for example Methocel E5 or E 15 (Dow Chemical Ltd) or Pharmacoat 606 (Shin Etsu).

A film coating having a retarding action may consist of a water-insoluble but water permeable polymer which, as a diffusion barrier, not only bring about a lag time in the beginning but also affect the swelling behavior of the core over a prolongered period as a result of the initially altered water permeation. Preferred water insoluble polymers are water-insoluble derivatives of methacrylic acid, for example methyl/ethyl acrylate, such as Eudragit® RS or RL and Eudragit® NE (brand names, rohm Pharma GmbH) and mixtures thereof.

Other representative film formers include include cellulose acetate butyrate, cellulose acetate propionate, cellulose propionate, HPMC, carrageenan, cellulose nitrate, hydrophilic cellulosic agents, hydroxypropylcellulose, methylcellulose, hydroxyethylcellulose, ethylcellulose, polyvinyl acetate and latex dispersions, poly-acids, enteric polymers, polysaccharides, acacia, tragacanth, guar gum, gelatin, proteins, albumin, polylactic acid, biodegradable polymers, polyglutamic acid and combinations thereof.

The film coating may also contain excipients customary in film-coating procedures, such as light-protective pigments, for example iron oxide, in an amount from about 40-80%, or titanium oxide, in an amount of about 100-150%, anti-adhesive agents, for example talc, in an amount from about 50-200%, and also suitable plasticiers, matched to the polymer, of the polyethylene glycol series, for example PEG 400 or PEG 6,000 or triethyl citrate in the case of films based on methylacrylic acid derivatives, such as Eudragit® RS or RL and Eudragit® NE, in an amount from about 30-60% (percentages in each case are based on the dry coating substance). When aqueous dispersions of the Eudragit® types are used, then, for example, Tween 80 is necessary as an aggregation inhibitor.

The formulations of the present invention may contain opaquants. As used herein, the term “opaquant” is intended to mean a compound used to render a capsule or a tablet coating opaque. Opaquants may be used alone or in combination with a colorant. Such compounds include, by way of example and without limitation, titanium dioxide and the like.

The formulations of the present invention can also release the active ingredient(s) only, or preferentially, in a certain part of the intestinal tract in a delayed manner. Examples of embedding compositions which can be used include polymeric substances and waxes.

A variety of components or compounds can be used to aid in the preparation of suitable dosage forms for the present invention. Such components or compounds include, without limitation, an acidifying agent, alkalinizing agent, adsorbent, antifungal preservative, antioxidant, buffering agent, colorant, encapsulating agent, flavorant, stiffening agent, suppository base, sweetening agent, tablet antiadherent, tablet binder, tablet and capsule diluent, tablet coating agent, tablet direct compression excipient, tablet disintegrant, tablet glidant, tablet lubricant, tablet/capsule opaquant and tablet polishing agent.

As used herein, the term “acidifying agent” is intended to mean a compound used to provide acidic medium for product stability. Such compounds include, by way of example and without limitation, acetic acid, citric acid, fumaric acid, hydrochloric acid, and nitric acid and the like.

As used herein, the term “alkalinizing agent” is intended to mean a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium hydroxide, triethanolamine, and trolamine and the like.

As used herein, the term “adsorbent” is intended to mean an agent capable of holding other molecules onto its surface by physical or chemical (chemisorption) means. Such compounds include, by way of example and without limitation, powdered and activated charcoal and the like.

As used herein, the term “preservative” is intended to mean a compound used to prevent the growth of microorganisms. Such compounds include, by way of example and without limitation, benzalkonium chloride, benzethonium chloride, benzyl alcohol, cetylpyridinium chloride, chlorobutanol, phenol, phenylethyl alcohol, phenylmercuric nitrate and thimerosal and the like.

As used herein, the term “antioxidant” is intended to mean an agent which inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and the like.

As used herein, the term “buffering agent” is intended to mean a compound used to resist change in pH upon dilution or addition of acid or alkali. Such compounds include, by way of example and without limitation, potassium metaphosphate, potassium phosphate, monobasic sodium acetate and sodium citrate anhydrous and dihydrate and the like.

As used herein, the term “tablet direct compression excipient” is intended to mean a compound used in direct compression tablet formulations. Such compounds include, by way of example and without limitation, dibasic calcium phosphate (e.g., Ditab), phosphor spray dried, or anhydrous lactose, microcrystalline cellulose, (AVICEL™), dextran (EMDEX™), sucrose (NUTAB™) and others know to those of ordinary skill in the art.

As used herein, the term “tablet glidant” is intended to mean agents used in tablet and capsule formulations to reduce friction during tablet compression. Such compounds include, by way of example and without limitation, colloidal or fumed silica, magnesium stearate, cornstarch, and talc and the like. Other glidants or lubricants include calcium stearate, mineral oil, stearic acid, hydrogenated vegetable oil, benzoic acid, poly(ethylene glycol), NaCl, PRUV™, zinc stearate and the like.

As used herein, the term “tablet anti-adherents” is intended to mean agents which prevent the sticking of table formulation ingredients to punches and dies in a tableting machine during production. Such compounds include, by way of example and without limitation, magnesium stearate, corn starch, silicone dioxide, talc and the like.

As used herein, the term “tablet polishing agent” is intended to mean a compound used to impart an attractive sheen to coated tablets. Such compounds include, by way of example and without limitation, carnauba wax, and white wax and the like.

As used herein, the term “tablet binders” is intended to mean substances used to cause adhesion of powder particles in table granulations. Such compounds include, by way of example and without limitation, acacia, alginic acid, carboxymethylcellulose sodium, compressible sugar (e.g., NuTab), ethylcellulose, gelatin, liquid glucose, methylcellulose, povidone and pregelatinized starch and the like. The melting and/or softening point temperatures of these binders usually rise with increase of their molecular weights. Binders having a melting or softening point temperature greater than about 150° C. may require use of a plasticizer during preparation of a suitable dosage form such that the binder melting or softening point temperature will be lowered below 150° C. The binder is generally in the form of a powder, granules, flakes or heat-molten liquid.

Plasticizers may be useful in the formulations. As used herein, the term “plasticizer” includes all compounds capable of plasticizing a binder. The plasticizer should be able to lower the melting temperature or glass transition temperature (softening point temperature) of the binder. Plasticizers, such as low molecular weight PEG, generally broaden the average molecular weight of the binder thereby lowering its glass transition temperature or softening point. Plasticizers also generally reduce the viscosity of a polymer. It is possible the plasticizer will impart some particularly advantageous physical properties to the formulation of the invention.

Plasticizers can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate esters, triacetin, propylene glycol phthalate esters, phosphate esters, sebacate esters, glycol derivatives, fatty acid esters, and glycerin.

Such plasticizers can also be ethylene glycol, 1,2-butylene glycol, 2,3-butylene glycol, styrene glycol, diethylene glycol, dipropylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, dimethylsebacate, di-2-ethylhexylsebacate, tricresyl phosphate, triethyl phosphate, triphenyl phosphate, acetylated monoglycerides, mineral oil, castor oil, glyceryl triacetate, butyl stearate, glycerol monostearate, butoxyethyl stearate, stearyl alcohol, cyclohexyl ethyl phthalate, cyclohexyl methyl dibutylphthalate, diethyl phthalate, dibutyl phthalate, diisopropyl phthalate, dimethyl phthalate, dioctyl phthalate, acetyl tributyl citrate, triethyl citrate, acetyl triethyl citrate, tributyl citrate and allyl glycolate. All such plasticizers are commercially available from sources such as Aldrich or Sigma Chemical Co. or Morflex, Inc. It is contemplated and within the scope of the invention, that a combination of plasticizers may be used in the present formulation.

When the controlled release dosage form is a polymer matrix, pore forming agents can be included in an amount of between 0.01% and 90% weight to volume, to increase matrix porosity and pore formation during the production of the matrices.

A preferred formulation contains at least sodium 4-phenylbutyrate, or an ester, hydrate, or prodrug thereof, hydroxypropylmethylcellulose, and lactose. Further additives, such as described above, can be added in amounts necessary in order to obtain the desired formulation, in accordance with the present invention.

D. Dosage Forms

The compounds and formulations of the present invention can be administered in any of the known dosage forms standard in the art, including without limitation, solid dosage form, semi-solid dosage form, or liquid dosage form, as well as subcategories of each of these forms.

Solid dosage forms for oral administration include capsules, caplets, tablets, pills, powders, lozenges, and granules. As used herein, the term “tablet” is intended to include compressed tablets, coated tablets, matrix tablets, osmotic tablets, and other forms known in the art, as more fully described above. As used herein, the term “capsule” is intended to include capsules in which the body of the capsule disintegrates after ingestion to release particulate contents which exhibit the desired sustained-release behavior, and also capsules for which the body of the capsule remains substantially intact during its residence in the GI tract. Multiparticulate dosage forms are also contemplated, wherein dosage form contain a multiplicity of particles whose totality represents the intended therapeutically useful dose of 4-PBA.

In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Semi-liquid dosage forms include those dosage forms that are too soft in structure to qualify for solids, but to thick to be counted as liquids. These include creams, pastes, ointments, gels, lotions, and other semisolid emulsions containing the active compound of the present invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Dosage forms for topical or transdermal administration of a compound of this invention include ointments, pastes, creams, lotions, gels, powders, solutions, sprays, inhalants or patches, optionally mixed with degradable or nondegradable polymers. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

Formulations containing compounds of the invention may be administered through the skin by an appliance such as a transdermal patch. Patches can be made of a matrix such as polyacrylamide, polysiloxanes, or both and a semi-permeable membrane made from a suitable polymer to control the rate at which the material is delivered to the skin. Other suitable transdermal patch formulations and configurations are described in U.S. Pat. Nos. 5,296,222 and 5,271,940, as well as in Satas, D., et al, “Handbook of Pressure Sensitive Adhesive Technology, 2^{nd} Ed.”, Van Nostrand Reinhold, 1989: Chapter 25, pp. 627-642.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

The formulations may be prepared by any of the methods well known in the art of pharmacy. All methods include the step of bringing into association a compound of the invention or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

The amount of therapeutic compound incorporated into the present formulations is selected according to known principles of pharmacy, clinical medicine and pharmacology. A therapeutically effective amount of therapeutic compound is specifically contemplated. By the term “therapeutically effective amount,” it is understood that, with respect to, for example, pharmaceuticals, a pharmaceutically effective amount is contemplated. A pharmaceutically effective amount is the amount or quantity of a drug or pharmaceutically active substance which is sufficient to elicit the required or desired therapeutic response, or in other words, the amount which is sufficient to elicit an appreciable biological response when administered to a patient.

The amount of sodium 4-phenylbutyrate added to the controlled-release formulations of the present invention can be from about 0.01 μg to about 1,000 g, and include but are not limited to amounts of about 0.01 mg, about 0.1 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, about 1,000 mg, and amounts between any of the values included herein (e.g., an amount from about 20 mg to about 800 mg). Amounts given are amounts per tablet or dose prepared. In one embodiment, the amount of sodium 4-phenylbutyrate, or its ester, hydrate, or prodrug, is from about 50 mg to about 800 mg per tablet or dose.

The therapeutic compound is generally used in finely divided form, i.e. powder or granulate so as to increase the dissolution rate. It is preferable to use a finely powdered therapeutic compound to increase the dissolution rate, more preferably, the therapeutic compound being capable of allowing not less than 80%, desirably not less than 90%, of it to pass through a 100 mesh (150 microns) screen. The amount of therapeutic compound to be incorporated ranges usually from about 0.1 to 50%, preferably about 1 to 25% by weight based on the composition, and the ratio may be suitably modified depending on the therapeutic compound employed.

In one embodiment, the amount and type of hydroxypropylmethylcellulose (HPMC) added to the controlled-release formulations of the present invention include but are not limited to any suitable, commercially available or readily synthesized type (e.g., 2208, USP XXII of 100 or 400 cps). The amount of HPMC added to the controlled-release formulations of the present invention can be any amount suitable to allow for the desired slow-release (extended release), long action formulation of the invention to be achieved. The amount of HPMC added to formulations described herein thus includes but is not limited to amounts from about 0.1 mg to about 1000 mg per tablet or dose. This includes but is not limited to about 0.1 mg, about 0.2 mg, about 0.3 mg, about 0.4 mg, about 0.5 mg, about 1.0 mg, about 2.0 mg, about 3.0 mg, about 4.0 mg, about 5.0 mg, about 6.0 mg, about 7.0 mg, about 8.0 mg, about 9.0 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg, about 500 mg, about 600 mg, about 700 mg, about 800 mg, about 900 mg, and about 1,000 mg, as well as amounts between any of the values included herein (e.g., an amount from about 120 mg to about 180 mg).

According to one embodiment, the amount and type of lactose added to the controlled-release formulations of the present invention include but are not limited to any commercially available or readily synthesized type of lactose suitable for use in pharmaceutical formulations. The amount of lactose added to the controlled-release formulations of the present invention can be any amount suitable to allow for the desired slow-release (extended release), long action formulation of the invention to be achieved. The amount of lactose added to formulations described herein thus includes but is not limited to amounts from about 0.1 mg to about 1000 mg per tablet or dose. This includes but is not limited to about 0.1 mg, about 0.2 mg, about 0.3 mg, about 0.4 mg, about 0.5 mg, about 1.0 mg, about 2.0 mg, about 3.0 mg, about 4.0 mg, about 5.0 mg, about 6.0 mg, about 7.0 mg, about 8.0 mg, about 9.0 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg, about 500 mg, about 600 mg, about 700 mg, about 800 mg, about 900 mg, and about 1,000 mg per tablet or dose, as well as amounts between any of the values included herein (e.g., an amount from about 120 mg to about 180 mg).

In a particular embodiment of the present invention, a controlled-release tablet formulation of sodium 4-phenylbutyrate contains at least about 150 to about 275 mg of sodium 4-phenylbutyrate, at least about 100 mg to about 200 mg of hydroxypropylmethylcellulose, and at least about 200 mg to about 300 mg of lactose, per tablet or dose. Optionally, additives such as described above and known in the pharmaceutical formulation arts can be included in the tablet or dose formulation, including any or all combinations of microcrystalline cellulose, such as Avicel® PH 102, hardened vegetable oil (e.g., talcum), magnesium stearate, and/or silicon dioxide. In such an embodiment, in accordance with the present invention, the amounts of microcrystalline cellulose which can be added are from about 10 mg to about 500 mg per tablet or dose; the amount of hardened vegetable oil added can be from about 1 mg to about 100 mg per tablet or dose; the amount of lubricant, such as magnesium stearate, added can be from about 0.5 mg to about 100 mg per tablet or dose, and the amount of optionally added silicon dioxide (SiO₂) can be from about 0.1 mg to about 100 mg per tablet or dose.

In one example of a controlled-release tablet or dose formulation in accordance with the present invention, a tablet is prepared which contains from about 1 mg to about 1000 mg of sodium phenylbutyrate, or other suitable salts, esters, and prodrugs thereof; from about 1 mg to about 1,000 mg of hydroxypropylmethylcellulose; from about 0 (i.e., none) to about 1,000 mg of lactose; from about 0.1 to about 500 mg of microcrystalline cellulose; from about 1 mg to about 100 mg of hardened vegetable oil; from about 0.5 mg to about 100 mg of magnesium stearate; and from about 0.1 mg to about 100 mg of highly dispersed silicon dioxide.

Materials to be incorporated in the present formulation can be pretreated to form granules. This process is known as granulation. As commonly defined, “granulation” is any process of size enlargement whereby small particles are gathered together into larger, permanent aggregates to yield a free-flowing composition having a suitable consistency. Such granulated compositions may have consistency similar to that of dry sand. Granulation may be accomplished by agitation in mixing equipment or by compaction, extrusion or agglomeration.

E. Administration

The compounds of the invention are preferably administered by any appropriate administration route, for example, orally, parenterally, intravenously, intradermally, intramuscularly, subcutaneously, sublingually, transdermally, bronchially, pharyngolaryngeal, intranasally, topically such as by a cream or ointment, rectally, intraarticular, intracisternally, intrathecally, intravaginally, intraperitoneally, intraocularly, by inhalation, bucally or as an oral or nasal spray. The route of administration may vary, however, depending upon the condition and the severity of the diabetic vascular disease or ocular inflammation.

The precise amount of compound administered to a host or patient will be the responsibility of the attendant physician. However, the dose employed will depend on a number of factors, including the age and sex of the patient, the precise disorder being treated, and its severity. In accordance with the compositions of the present invention, a dose range of from about 0.001 mg/kg per day to about 2500 mg/kg per day is typical. Preferably, the dose range is from about 0.1 mg/kg per day to about 1000 mg/kg per day. More preferably, the dose range is from about 0.1 mg/kg per day to about 500 mg/kg per day, including 1 mg/kg, 2 mg/kg, 5 mg/kg, 10 mg/kg, 15 mg/kg, 20 mg, kg, 25 mg/kg, 30 mg/kg, 35 mg/kg, 40 mg/kg, 45 mg/kg, 50 mg/kg, 100 mg/kg, 200 mg/kg, 300 mg/kg, 400 mg/kg, 500 mg/kg per day, and values between any two of the values given in this range.

The dose range for humans is generally from about 0.005 mg to 100 g/day. Alternatively, the dose range in accordance with the present invention is such that the blood serum level of compounds of the present invention is from about 0.01 μM to about 100 μM, and preferably from about 0.1 μM to about 100 μM. Suitable values of blood serum levels in accordance with the present invention include but are not limited to about 0.01 μM, about 0.1 μM about 0.5 μM, about 1 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM and about 100 μM, as well as any blood serum level that falls within any two of these values (e.g., between about 10 μM and about 60 μM). Tablets or other forms of dosage presentation provided in discrete units may conveniently contain an amount of one or more of the compounds of the invention which are effective at such dosage rages, or ranges in between these ranges.

The pharmaceutical formulation of the present invention will maintain therapeutically beneficial blood levels of 4-PBA over an extended period of time. Suitable time periods of controlled release include but are not limited to about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours and about 32 hours, as well as any time period that falls between any of these periods. In one embodiment, the pharmaceutical formulation provides therapeutically beneficial blood levels of 4-PBA for about 6 hours. In another embodiment, the pharmaceutical formulation provides therapeutically beneficial blood levels of 4-PBA for about 12 hours. In a further embodiment, the pharmaceutical formulation provides therapeutically beneficial blood levels of 4-PBA for about 24 hours.

According to one embodiment of the present invention, the effective dose upon twice daily administration amounts to about 40 to about 2,000 mgs per day. Preferably, the effective dose upon twice daily administration amounts to about 500 mg per day.

F. Therapeutic Uses

The formulations contain a release-controlling agent and 4-phenylbutyric acid compounds, including the pharmaceutically acceptable salts, esters, and prodrugs of 4-phenylbutyric acid, can be used for a number of therapeutic applications. Notably, the control-release formulations of the present invention can be used to treat a variety of diseases and disorders, including urea cycle disorders, phenylketonuria, alpha-1-antitrypsin deficiency disorders, hematology/blood-related disorders and diseases, neoplastic disorders, viral disorders, histone deacetylation diseases or disorders, cancer or neoplastic diseases, betaglobin disorders, and diseases or disorders of the nervous system, such as CNS diseases or disorders.

(i) Urea Cycle Disorders

Urea cycle disorders are genetic disorders caused by a deficiency of one of the enzymes in the urea cycle which is responsible for removing ammonia from the blood stream. The urea cycle involves a series of biochemical steps in which nitrogen, a waste product of protein metabolism, is removed from the blood and converted to urea. Normally, the urea is transferred into the urine and removed from the body. In urea cycle disorders, the nitrogen accumulates in the form of ammonia, a highly toxic substance, and is not removed from the body. Ammonia then reaches the brain through the blood, where it causes irreversible brain damage and/or death.

Urea cycle disorders are included in the category of inborn errors of metabolism. Urea cycle disorders which can be treated with the controlled-release formulations according to the present invention include but are not limited to carbamyl phosphate synthetase (CPS) deficiency, N-acetylglutamate synthetase (NAGS) deficiency, ornithine transcarbamylase (OTC) deficiency, argininosuccinic acid synthetase deficiency (ASD; citrullinemia), argininosuccinase acid lyase deficiency (ALD: argininosuccinic aciduria), and arginase (AG) deficiency.

(ii) Phenylketonuria

Phenylketonuria, or PKU, is a hereditary disease caused by a lack of a liver enzyme (phenylalanine hydroxylase) required to digest phenylalanine, an amino acid commonly found in protein-containing foods such as meat, cow's milk, infant formulas, and breast milk. Included in these phenylalanine-deficiency-related disorders, in addition to phenylketonuria (PKU), are non-PKU hyperphenylalaninemia (non-PKU HPA), and variant PKU. Classic PKU is due to a complete or near-complete deficiency of phenylalanine hydroxylase activity; without dietary restriction of phenylalanine, most children with PKU develop profound and irreversible mental retardation. Non-PKU HPA has been associated with a much lower risk of impaired cognitive development in the absence of treatment. Variant PKU is intermediate between PKU and non-PKU HPA. PKU disorders which can be treated with the controlled-release formulations of the present invention include classic phenylketonuria, variant phenylketonuria, and non-phenylketonuria hyperphenylalaninemia.

(iii) Alpha-1-antitrypsin Deficiency Disorders

The present invention also relates to methods for the use of the controlled-release formulations of 4-phenylbutyric acid and its pharmaceutically acceptable derivatives (salts, esters, and prodrugs) as described herein in the treatment of alpha-1-antitrypsin deficiencies in subjects. Alpha-1-antitrypsin deficiency disorders are those diseases and disorders which result from a deficiency of the protein alpha-1-antitrypsin in the bloodstream. Included in these disorders which can be suitably treated, prevented, or inhibited with the controlled-release formulations of the present invention are liver-disease associated with or caused by alpha-1-antitrypsin deficiency, including alpha-1-antitrypsin deficiency caused by a PiZ (protease inhibitor type Z) mutation. Additionally, the present invention also provides methods for the treatment, inhibition, and/or prevention of emphysema and/or lung-damage (such as loss of elasticity of the lungs) in subjects with alpha-1-antitrypsin deficiency, including those alpha-1-antitrypsin deficiencies caused by a PiZ mutation.

The methods for treatment of alpha-1-antitrypsin deficiency in a subject by the administration of a controlled-release formulation of the present invention are envisioned to include administering to a subject with alpha-1-antitrypsin deficiency an alpha-1-antitrypsin secretion stimulation amount of 4-phenylbutyric acid, or its pharmaceutically acceptable salts, esters or prodrugs, in a controlled-release formulation as described herein. Also included within this aspect of the present invention is to provide a method for correcting alpha-1-antitrypsin deficiency by detecting the presence of alpha-1-antitrypsin deficiency in a subject, stimulating secretion of alpha-1-antitrypsin by administering an alpha-1-antitrypsin stimulating amount of 4-phenylbutyric acid, or its pharmaceutically acceptable salts, esters, or prodrugs in a controlled-release formulation as described herein, and monitoring the alpha-1-antitrypsin levels during and after treatment. Finally, yet another aspect of the present invention with regard to alpha-1-antitrypsin deficiency is a method for stimulating the secretion of alpha-1-antitrypsin by a cell by contacting a cell containing a protease inhibitor type Z mutation with an alpha-1-antitrypsin secretion stimulating amount of 4-phenylbutyric acid, or its pharmaceutically acceptable salts, esters, or prodrugs in a controlled-release formulation as described herein, and monitoring the alpha-1-antitrypsin levels during and after treatment.

(iv) Hematology Disorders/Blood Disorders

Methods for the administration of the controlled-release formulation compositions of the present invention are also preferably used for the treatment of blood disorders such as hemoglobinopathies (e.g. sickle cell anemia, thalassemia), and cell proliferative disorders such as viral-induced malignancies (e.g. latent virus infections) and cytopenia including red and white blood cell anemia, leukopenia, neutropenia and thrombocytopenia.

Another embodiment of the invention is directed to methods for the treatment of patients with blood disorder by the administration of one or more controlled-release compositions/formulations of the present invention which comprise at least 4-phenylbutyric acid, or a pharmaceutically acceptable salt, ester, or prodrug thereof. Compositions to be administered contain a therapeutically effective amount of 4-phenylbutyric acid, or a pharmaceutically acceptable salt, ester, or prodrug thereof (such as sodium phenylbutyrate), and a release-controlling agent. A therapeutical effective amount is that amount which has a beneficial effect to the patient by alleviating one or more symptoms of the disorder or simply reduce premature mortality. For example, a beneficial effect may be a decrease in pain, a decrease in duration, frequency or intensity of crises, an increased hematocrit, an improved erythropoiesis, a reduced or eliminated necessity for chelation therapy, an increased reticulocyte count, an increased peripheral blood flow, a decreased hemolysis, decreased fatigue or an increased strength. Preferably, a therapeutic amount is that amount of chemical compound or agent that stimulates or enhances the expression of non-adult globin such as embryonic or fetal globin, or the proliferation of embryonic, fetal or adult globin expressing cells. A therapeutically effective amount for continuous therapy is typically greater than a therapeutically amount that is effective in pulsed therapy.

(v) Infectious Diseases

Another embodiment of the invention is directed to methods for the treatment of a patient with an infectious disease or disorder by administering a therapeutically effective composition of the controlled-release formulation of 4-phenylbutyric acid (or its pharmaceutically acceptable salts, ester, and prodrugs) and a release-control agent.

Treatable infectious diseases include bacterial infections such as sepsis and pneumonia, infections caused by bacterial pathogens such as, for example, Pneumococci, Streptococci, Staphylococci, Neisseria, Chlamydia, Mycobacteria, Actinomycetes and the enteric microorganisms such as enteric Bacilli; viral infections caused by, for example, a hepatitis virus, a retrovirus such as HIV, an influenza virus, a papilloma virus, a herpes virus (HSV I, HSV II, EBV), a polyoma virus, a slow virus, paramyxovirus and corona virus; parasitic diseases such as, for example, malaria, trypanosomiasis, leishmania, amebiasis, toxoplasmosis, sarcocystis, pneumocystis, schistosomiasis and elephantitis; and fungal infections such as candidiasis, phaeohyphomycosis, aspergillosis, mucormycosis, cryptococcosis, blastomycosis, paracoccidiodomycosis, coccidioidomycosis, histomycosis, actinomycosis, nocardiosis and the Dematiaceous fungal infections.

(vi) Neoplastic Disorders

A further embodiment of the invention is directed to methods for the treatment of a patient with a neoplastic disorder by administering a therapeutically effective composition of the controlled-release formulation of 4-phenylbutyric acid (or its pharmaceutically acceptable salts, ester, and prodrugs).

The neoplastic disorder may be any disease or malady which could be characterized as a neoplasm, a tumor, a malignancy, a cancer or a disease which results in a relatively autonomous growth of cells. Neoplastic disorders prophylactically or therapeutically treatable with compositions of the invention include small cell lung cancers and other lung cancers, rhabdomyosarcomas, chorio carcinomas, glioblastoma multiformas (brain tumors), bowel and gastric carcinomas, leukemias, bladder cancers, ovarian cancers, prostate cancers, osteosarcomas or cancers which have metastasized. Diseases of the immune system which are treatable by these compositions include the non-Hodgkin's lymphomas including the follicular lymphomas, Burlitt's lymphoma, adult T-cell leukemias and lymphomas, hairy-cell leukemia, acute myelogenous, lymphoblastic or other leukemias, chronic myelogenous leukemia, and myelodysplastic syndromes. Additional diseases treatable by the compositions include virally-induced cancers wherein the viral agent is EBV, HPV, HIV, CMV, HTLV-1 or HBV, breast cell carcinomas, melanomas and hematologic melanomas, pancreatic cancers, liver cancers, stomach cancers, colon cancers, bone cancers, squamous cell carcinomas, neurofibromas, testicular cell carcinomas and adenocarcinomas, endometrial cancer, kidney cancer, leukemia, melanoma, non-Hodgkin's lymphoma, skin cancer or thyroid cancer.

In a preferred embodiment, the compositions and methods of the present invention are useful for the treatment or prevention or prostate cancer. Although several cell types are found in the prostate, over 99% of prostate cancers develop from the glandular cells (i.e., adenocarcinoma). The present invention can be used to treat prostate cancer, or slow its progression, at all stages. In general, prostate cancer is characterized as localized, regional or metastatic. Localized prostate cancer may be (i) Stage I or A or T1 (where a tumor that cannot be felt (nonpalpable)) or (ii) Stage II or B or T2 (where a tumor that can be felt (palpable) but is confined to the prostate gland). Regional prostate cancer is described as (i) Stage III or C or T3 (where a tumor that has grown through the prostate capsule, perhaps into the seminal vesicles) or (ii) T4 (where a tumor that has grown into nearby muscles and organs. Metastatic prostate cancer is typically described as Stage 1V or D and N+ or M⁺: tumors that have metastasized to the regional (pelvic) lymph nodes (N+) or more distant parts of the body (M+).

Neoplastic disorders that can be treated with the controlled release formulation of the present invention also include virus-induced tumors, malignancies, cancers or diseases which result in a relatively autonomous growth of cells. Neoplastic disorders include leukemias, lymphomas, sarcomas, carcinomas such as a squamous cell carcinoma, a neural cell tumor, seminomas, melanomas, germ cell tumors, undifferentiated tumors, neuroblastomas (which are also considered a carcinoma by some), mixed cell tumors or other malignancies.

Anti-neoplastic activity includes, for example, the ability to induce the differentiation of transformed cells including cells which comprise leukemias, lymphomas, sarcomas, neural cell tumors, carcinomas including the squamous cell carcinomas, seminomas, melanomas, neuroblastomas, mixed cell tumors, germ cell tumors, undifferentiated tumors, neoplasm due to infection (e.g. viral infections such as a human papilloma virus, herpes viruses including Herpes Simplex virus type I or II or Epstein-Barr virus, a hepatitis virus, a human T cell leukemia virus (HTLV) or another retrovirus) and other malignancies. Upon differentiation, these cells lose their aggressive nature, no longer metastasize, are no longer proliferating and eventually die and/or are removed by the T cells, natural killer cells and macrophages of the patient's immune system. The process of cellular differentiation is stimulated or turned on by, for example, the stimulation and/or inhibition of gene specific transcription. Certain gene products are directly involved in cellular differentiation and can transform an actively dividing cell into a cell which has lost or has a decreased ability to proliferate. An associated change of the pattern of cellular gene expression can be observed. To control this process includes the ability to reverse a malignancy. Genes whose transcriptional regulation are altered in the presence of compositions of the invention include the oncogenes myc, ras, myb, jun, fos, abl and src. The activities of these gene products as well as the activities of other oncogenes are described in J. D. Slamon, et al. (Science, 224: pp. 256-62 (1984)).

Another example of anti-neoplastic activity includes the ability to regulate the life cycle of the cell, the ability to repress angiogenesis or tissue regeneration through the blockade or suppression of factor activity, production or release, the ability to regulate transcription or translation, or the ability to modulate transcription of genes under angiogenesis, growth factor or hormonal control. These activities are an effective therapy particularly against prostatic-neoplasia and breast carcinomas. Additional anti-neoplastic activities include the ability to regulate the cell cycle for example by effecting time in and passage through S phase, M phase, G₁ phase or G₀ phase, the ability to increase intracellular cAMP levels, the ability to inhibit or stimulate histone acetylation, the ability to methylate nucleic acids and the ability to maintain or increase intracellular concentrations of anti-neoplastic agents.

In another embodiment of the invention, compositions may be administered in combination with other anti-neoplastic agents or therapies to maximize the effect of the compositions in an additive or synergistic manner. Cytokines which may be effective in combination with the compositions include growth factors such as B cell growth factor (BCGF), fibroblast-derived growth factor (FDGF), granulocyte/macrophage colony stimulating factor (GM-CSF), granulocyte colony stimulating factor (G-CSF), macrophage colony stimulating factor (M-CSF), epidermal growth factor (EGF), platelet derived growth factor (PDGF) nerve growth factor (NGF), stem cell factor (SCF), and transforming growth factor (TGF). These growth factors plus a composition may further stimulate cellular differentiation and/or the expression of certain MHC antigens or tumor specific antigens. For example, BCGF plus a composition may be effective in treating certain B cell leukemias. NGF plus a composition may be useful in treating certain neuroblastomas and/or nerve cell tumors. In a similar fashion, other agents such as differentiating agents may be useful in combination with a composition to prevent or treat a neoplastic disorder. Other differentiating agents include B cell differentiating factor (BCDF), erythropoietin (EPO), steel factor, activin, inhibin, the bone morphogenic proteins (BMPs), retinoic acid or retinoic acid derivatives such as retinol, the prostaglandins, and TPA.

Alternatively, other cytokines and related antigens in combination with a composition may also be useful to treat or prevent neoplasia. Potentially useful cytokines include tumor necrosis factor (TNF), the interleukins (IL-1, IL-2, IL-3, etc.), the interferon proteins (IFN) IFN-α, IFN-β, and IFN-γ, cyclic AMP including dibutyryl cyclic AMP, hemin, hydroxyurea, hypoxanthine, glucocorticoid hormones, dimethyl sulfoxide (DMSO), and cytosine arabinoside, and anti-virals such as acyclovir and gemciclovirs. Therapies using combinations of these agents would be safe and effective against malignancies and other forms of cancer. Combinations of therapies may also be effective in inducing regression or elimination of a tumor or some other form of cancer such as pulsed compositions plus radiation therapy, toxin or drug conjugated antibody therapy using monoclonal or polyclonal antibodies directed against the transformed cells, gene therapy or specific anti-sense therapy. Effects may be additive, logarithmic, or synergistic, and methods involving combinations of therapies may be simultaneous protocols, intermittent protocols or protocols which are empirically determined.

Another embodiment of the invention provides methods for the administration of the controlled-release compositions of the present invention for the treatment of neoplastic disorders by augmenting conventional chemotherapy, radiation therapy, antibody therapy, and other forms of therapy. Compositions containing chemical compounds in combination with chemotherapeutic agents, enhance the effect of the chemotherapeutic agent alone. Compositions decrease the expression or activity of proteins responsible for lowering the intra-cellular concentration of chemotherapeutic agents. Proteins responsible for resistance to drugs and other agents, the multi-drug resistance (MDR) proteins, include the P-glycoprotein (Pgp) encoded by the mdr-1 gene. Consequently, conventional drugs for the treatment of neoplastic disorders accumulate at higher concentrations for longer periods of time and are more effective when used in combination with the compositions herein. Some conventional chemotherapeutic agents which would be useful in combination therapy with compositions of the invention include the cyclophosphamide such as alkylating agents, the purine and pyrimidine analogs such as mercapto-purine, the vinca and vinca-like alkaloids, the etoposides or etoposide like drugs, the antibiotics such as deoxyrubocin and bleomycin, the corticosteroids, the mutagens such as the nitrosoureas, antimetabolites including methotrexate, the platinum based cytotoxic drugs, the hormonal antagonists such as antiinsulin and antiandrogen, the antiestrogens such as tamoxifen an other agents such as doxorubicin, L-asparaginase, dacarbazine (DTIC), amsacrine (mAMSA), procarbazine, hexamethylmelamine, and mitoxantrone. The chemotherapeutic agent could be given simultaneously with the compounds of the invention or alternately as defined by a protocol designed to maximize drug effectiveness, but minimize toxicity to the patient's body.

(vii) Histone Deacetylation Disorders

Histone deacetylase is a metallo-enzyme with zinc at the active site. Compounds having a zinc-binding moiety, such as, for example, a hydroxamic acid group, can inhibit histone deacetylase. Histone deacetylase inhibition can repress gene expression, including expression of genes related to tumor suppression. Accordingly, inhibition of histone deacetylase can provide an alternate route for hematological disorders, e.g., hemoglobinopathies, and genetic related metabolic disorders, e.g., cystic fibrosis and adrenoleukodystrophy.

In one aspect of the present invention, methods are provided for treatment of histone deacetylation-related diseases or disorders in a subject by the administration of a controlled-release formulation of the present invention which include administering to a subject with a histone deacetylation disease or disorder, such as cystic fibrosis (CF), a neurodegenerative disease or cancer, a histone deacetylase stimulating amount of 4-phenylbutyric acid, or its pharmaceutically acceptable salts, esters or prodrugs, in a controlled-release formulation as described herein. Also included within this aspect of the present invention is to provide a method for correcting histone deacetylase disorders by detecting the presence of histone deacetylase inhibition in a subject, stimulating secretion of histone deacetylase by administering a histone deacetylase stimulating amount of 4-phenylbutyric acid, or its pharmaceutically acceptable salts, esters, or prodrugs in a controlled-release formulation as described herein, and monitoring the histone deacetylase levels during and after treatment.

(vii) Abnormal Protein Localization or Aggregation Disorders

A further embodiment of the invention is directed to methods for the treatment of a patient with a disorder characterized by abnormal protein localization and/or aggregation, by administering of a therapeutically effective composition of the controlled-release formulation of 4-phenylbutyric acid (or its pharmaceutically acceptable salts, ester, and prodrugs) and a release-control agent.

A wide range of inherited pathological conditions involve abnormal localization and/or aggregation of protein as a central component, including alpha-1-antitrypsin deficiency, Alzheimer's disease, prion diseases, familial Parkinsonism, familial forms of amyotrophic lateral sclerosis, spinocerebellar diseases, lysosomal storage diseases.

Acquired diseases may also involve protein aggregation, such as acquired ischemic conditions, in which aggregated proteins may also contribute to tissue injury, has also been proposed.

(ix) Betaglobin Disorders

Betaglobin is a polypeptide subunit of hemoglobin A, which is the principle oxygen carrier in the blood. Diseases and disorders which are known as betaglobulin disorders and are characterized by the production of either abnormal betaglobin or one of the 2 betaglobin chains in hemoglobin, or alternatively by the production of no or insufficient amounts of betaglobin and hemoglobin A. Betaglobulin disorders which can be treated in accordance with the methods and controlled-release formulations of 4-phenylbutyric acid (or its pharmaceutically acceptable salts, esters, or prodrugs) as described in the present invention include sickle-cell anemia, beta-thalassemia and related thalassemias, and hyperlipoproteinemia.

(x) Diseases of the Nervous System

The language “diseases of the nervous system” is intended to include diseases of the nervous system whose onset, amelioration, arrest, or elimination is effectuated by the compounds described herein. Examples of types of diseases of the nervous system include demyelinating, dysmyelinating and degenerative diseases. Examples of locations on or within the subject where the diseases may originate and/or reside include both central and peripheral loci. As the term “disease” is used herein, it is understood to exclude, and only encompass maladies distinct from, neoplastic pathologies and tumors of the nervous system, ischemic injury and viral infections of the nervous system. Examples of types of diseases suitable for treatment with the methods and compounds of the instant invention are discussed in detail below.

Diseases of the nervous system fall into two general categories: (a) pathologic processes such as infections, trauma and neoplasma found in both the nervous system and other organs; and, (b) diseases unique to the nervous system which include diseases of myelin and systemic degeneration of neurons.

Of particular concern to neurologists and other nervous system practitioners are diseases of: (a) demyelination which can develop due to infection, autoimmune antibodies, and macrophage destruction; and, (b) dysmyelination which result from structural defects in myelin.

Diseases of neurons can be the result of: (a) aberrant migration of neurons during embryogenesis and early stage formation; or (b) degenerative diseases resulting from a decrease in neuronal survival, such as occurs in, for example, Alzheimer's disease, Parkinson's disease, Huntington's disease, motor neuron disease, ischemia-related disease and stroke, and diabetic neuropathy.

(a) Demyelinating Diseases

Primary demyelination is a loss of myelin sheaths with relative preservation of the demyelinated axons. It results either from damage to the oligodendroglia which make the myelin or from a direct, usually immunologic or toxic attack on the myelin itself. Secondary demyelination, in contrast, occurs following axonal degeneration. The demyelinating diseases are a group of CNS conditions characterized by extensive primary demyelination. They include multiple sclerosis and its variants and perivenous encephalitis. There are several other diseases in which the principal pathologic change is primary demyelination, but which are usually conveniently classified in other categories such as inborn errors of metabolism, the leukodystrophies, viral disease (progressive multifocal leukoencephalopathy PM), as well as several other rare disorders of unclear etiology.

Multiple sclerosis is a disease of the central nervous system (CNS) that has a peak onset of 3040 years. It affects all parts of the CNS and causes disability related to visual, sensory, motor, and cerebellar systems. The disease manifestations can be mild and intermittent or progressive and devastating. The pathogenesis is due to an autoimmune attack on CNS myelin. The treatments available are symptomatic treating spasticity, fatigue, bladder dysfunction, and spasms. Other treatments are directed towards stopping the immunologic attack on myelin. These consist of corticosteroids such as prednisone and methylprednisolone, general immunosuppressants such as cyclophosphamide and azathioprine, and immunomodulating agents such as beta-interferon. No treatments are available to preserve myelin or make it resistant to attacks.

Acute Disseminated Encephalomyelitis is a disorder that usually occurs following a viral infection and is thought to be due to an autoimmune reaction against CNS myelin, resulting in paralysis, lethargy, and coma. It differs from MS by being a monophasic disease, whereas MS is characterized by recurrence and chronicity. Treatment typically consists of administration of steroids.

Acute Necrotizing Hemorrhagic Leukoencephalitis is a rare disease that is generally fatal. It is also thought to be mediated by autoimmune attack on CNS myelin that is triggered by a viral infection. Neurological symptoms develop abruptly with headache, paralysis and coma. Death usually follows within several days. Treatment is supportive.

Leukodystrophies are diseases of the white matter resulting from an error in the myelin metabolism that leads to impaired myelin formation. They are thought of as dysmyelinating diseases, and can become manifest at an early age.

Metachromatic Leukodystrophy is an autosomal recessive (inherited) disorder due to deficiency of the enzyme arylsulfatase, which leads to the accumulation of lipids. There is demyelination in the CNS and peripheral nervous system leading to progressive weakness and spasticity.

Krabbe's disease is also an inherited as autosomal recessive and due to deficiency of another enzyme, galctocerebroside beta-galactosidase.

Adrenoleukodystrophy and adrenomyeloneuropathy: affect the adrenal glad in addition to the nervous system.

(b) Degenerative Diseases

There is no good etiology or pathophysiology known for these diseases, and no compelling reason to assume that they all have a similar etiology. Diseases under this category have general similarities. They are diseases of neurons that tend to result in selective impairment, affecting one or more functional systems of neurons while leaving others intact.

Parkinson's disease results from the loss of dopaminergic neurons in the substantia nigra of the brain. It is manifested by slowed voluntary movements, rigidity, expressionless face and stooped posture. Several drugs are available to increase dopaminergic function such as levodopa, carbidopa, bromocriptine, pergolide, or decrease cholinergic function such as benztropine, and amantadine. Selegiline is a new treatment designed to protect the remaining dopaminergic neurons.

Spinocerebellar degenerations refers to a group of degenerative diseases that affects in varying degrees the basal ganglia, brain stem, cerebellum, spinal cord, and peripheral nerves. Patients present symptoms of Parkinsonism, ataxia, spasticity, and motor and sensory deficits reflecting damage to different anatomic areas and/or neuronal systems in the CNS.

Alzheimer's disease (AD) is a disease is characterized clinically by slow erosion of mental function, culminating in profound dementia. The diagnostic pathologic hallmark of AD is the presence of large numbers of senile plagues and neurofibrillary tangles in the brain especially in neocortex and hippocampus. Loss of specific neuron populations in these brain regions and in several subcortical nuclei correlates with depletion in certain neurotransmitters including acetylcholine. The etiology of AD is still unknown. To date a lot of research has focused on the composition and genesis of the B/A4 amyloid component of senile plagues. Alzheimer's disease is characterized clinically by the slow erosion of intellectual function with the development of profound dementia. There are no treatments that slow the progression.

Huntington disease (HD) is an autosomal dominant disorder of midlife onset, characterized clinically by movement disorder, personality changes, and dementia often leading to death in 15-20 years. The neuropathologic changes in the brain are centered in the basal ganglia. Loss of a class of projection neurons, called “spiny cells” because of their prominent dendritic spinous processes, is typical. This class of cells contains gamma-aminobutyric acid (GABA), substance P, and opioid peptides. Linkage studies have localized the gene for HD to the most distal band of the short arm of chromosome 4. No treatments are available that have been shown to retard progression of the disease. Experimental studies showing a similarity between neurons that are susceptible to N-methyl d-aspartate (NMDA) agonists and those that disappear in HD has led to encouraging speculation that NMDA antagonists might prove beneficial. Some recent studies suggest that a defect in brain energy metabolism might occur in HD and enhance neuronal vulnerability to excitotoxic stress.

Mitochondrial encephalomyopathies are a heterogeneous group of disorders affecting mitochondrial metabolism. These deficits could involve substrate transport, substrate utilization, defects of the Krebs Cycle, defects of the respiratory chain, and defects of oxidation/phosphorylation coupling. Pure myopathies vary considerably with respect to age at onset, course (rapidly progressive, static, or even reversible), and distribution of weakness (generalized with respiratory failure, proximal more than distal facioscapulohumeral, orbicularis and extraocular muscles with ptosis and progressive external ophthalmoplegia). Patients with mitochondrial myopathies complain of exercise intolerance and premature fatigue.

Degenerative diseases affecting motor neurons include such diseases such as amyotrophic lateral sclerosis (ALS), and spinal muscular atrophy (SMA). In a preferred embodiment, the compositions and methods of the present invention are used to treat degenerative diseases effective motor neurons, and more particularly to treat ALS or SMA.

Spinal muscular atrophy (SMA). SMA constitutes a group of neuromuscular disorders defined by a disease process limited to the anterior horn cell (AHC). (For a general review, see “The Spinal Muscular Atrophies,” by Theodore L. Munsat M. D., a long-time MDA Researcher. From Current Neurology, Chapter 3, Vol. 14, 1994, pp. 55-71). Weakness and wasting of the voluntary muscles is a central feature. Several common types of SMA most of which correlate with age of onset: (i) SMA type 1 (Werdnig-Hoffmann disease) is evident before birth or within the first few months of life; (ii) Type II usually appears between 3 and 15 months of age; (iii) SMA type III (Kugelberg-Welander disease) appears between 2 and 17 years of age; and (iv) Kennedy syndrome or progressive spinobulbar muscular atrophy may occur between 15 and 60 years of age. Congenital SMA with arthrogryposis (persistent contracture of joints with fixed abnormal posture of the limb) is a rare disorder. Most treatments for SMA are supportive in nature.

Amyotrophic lateral sclerosis (ALS). ALS, also known as “Lou Gehrig's disease,” is a progressive neurodegenerative disease that affects nerve cells in the brain and the spinal cord. As motor neurons degenerate, they can no longer send impulses to the muscle fibers that normally result in muscle movement. Early symptoms of ALS often include increasing muscle weakness, especially involving the arms and legs, speech, swallowing or breathing. When muscles no longer receive the messages from the motor neurons that they require to function, the muscles begin to atrophy (become smaller). Limbs begin to look “thinner” as muscle tissue atrophies. With voluntary muscle action progressively affected, patients in the later stages of the disease may become totally paralyzed. Approximately 50% of patients die within 34 years of diagnosis. The etiology and pathogenesis of ALS remain poorly understood. Present hypotheses include (i) altered glutamate metabolism; (ii) autoimmune mechanisms; (iii) oxidative stress; (iv) exogenous excitotoxins; and (iv) cytoskeletal abnormalities.

ALS is classified into three categories: (i) sporatic; (ii) familial; and (iii) environmental. Sporatic ALS is by far the most common form, accounting for 90 to 95% of cases. The cause of sporatic ALS is unknown. Familial ALS is genetically linked and accounts for 5 to 10% of all cases. Environmental ALS is thought to be associated with dietary factors. Sporatic ALS is itself subclassified into several disorders: (i) classical ALS—representing two thirds of all cases and involving both upper and lower motor neurons; (ii) progressive bulbar palsy—representing 25% of ALS cases and initially effecting the bulbar region; (iii) progressive muscular atrophy—representing 8010% of sporatic ALS cases and initially presenting with lower motor neuron symptoms; and (iii) primary lateral sclerosis—an extremely rare diagnosis which initially presents with upper motor neuron symptoms.

There is no cure or treatment that halts or reverses ALS. There is a single FDA approved drug, Rilutek® (2-amino-6-(trifluoromethoxybenzothiazole) (Aventis), that modestly slows the progression of ALS. Other drugs are being investigated in clinical trials.

(c) Peripheral Nervous System Disorders

The peripheral nervous system (PNS) consists of the motor and sensory components of the cranial and spinal nerves, the autonomic nervous system with its sympathetic and parasympathetic divisions, and the peripheral ganglia. It is the conduit for sensory information to the CNS and effector signals to the peripheral organs such as muscle. Contrary to the brain, which has no ability to regenerate, the pathologic reactions of the PNS include both degeneration and regeneration. There are three basic pathological degenerational processes which comprise the peripheral nervous system disorders: Wallerian degeneration, axonal degeneration, and segmental demyelination.

Some of the neuropathic syndromes include: acute ascending motor paralysis with variable sensory disturbance, examples being acute demyelinating neuropathics, infectious mononucleosis with polyneuritis, hepatitis and polyneuritis, toxic polyneuropathies; subacute sensorimotor polyneuropathy; examples of acquired axonal neurophathics include paraproteinemias, uremia diabetes, amyloidosis, connective tissue diseases and leprosy, while examples of inherited diseases include mostly chronic demyelination with hypertrophic changes, such as peroneal muscular atrophy, hypertrophic polyneuropathy and Refsum's diseases; chronic relapsing polyneuropathy; such as idiopathic polyneuritis porphyria, Beriberi and intoxications; mono or multiple neuropathy, such as pressure palsies, traumatic palsies, serum neuritis, zoster and leprosy.

G. Combination Therapy

The controlled-release formulations containing 4-phenylbutyrate compounds can be used in combination or alternation with a number of therapeutic compounds to effect a combination therapeutic approach to ameliorating diseases. In one embodiment, the controlled release formulation containing 4-phenylbutyric acid further containing a second therapeutic agent (i.e., the second therapeutic agent is also dispersed in the controlled release formulation).

For example, the 4-phenylbutyric acid controlled-release formulations can be used in combination with radiation and chemotherapy treatment, including induction chemotherapy, primary (neoadjuvant) chemotherapy, and both adjuvant radiation therapy and adjuvant chemotherapy in the treatment of cancers and tumors. In addition, radiation and chemotherapy are frequently indicated as adjuvants to surgery in the treatment of cancer. The goal of radiation and chemotherapy in the adjuvant setting is to reduce the risk of recurrence and enhance disease-free survival when the primary tumor has been controlled. Chemotherapy is utilized as a treatment adjuvant for lung and breast cancer, frequently when the disease is metastatic. Adjuvant radiation therapy is indicated in several diseases including lung and breast cancers. The 4-phenylbutyric acid compound containing controlled-release formulations also are useful following surgery in the treatment of cancer in combination with radio-and/or chemotherapy.

Chemotherapeutic agents that can be used in combination with the 4-phenylbutyric acid salts, esters or prodrugs in the controlled-release formulations of the present invention include those agents listed above in Section F of this Detailed Description, and further include but are not limited to, alkylating agents, antimetabolites, topoisomerase inhibitors, anti-tumor antibiotics, hormones and antagonists, microtubule stabilizers, radioisotopes, anti-inflammatories, antibacterial agents, plant alkyloids, antivirals, and antibodies, as well as natural products, and combinations thereof. For example, a compound of the present invention can be administered with antibiotics, such as doxorubicin and other anthracycline analogs, nitrogen mustards, such as cyclophosphamide, pyrimidine analogs such as 5-fluorouracil, cisplatin, hydroxyurea, and the like. As another example, in the case of mixed tumors, such as adenocarcinoma of the breast, where the tumors include gonadotropin-dependent and gonadotropin-independent cells, the compound can be administered in conjunction with leuprolide or goserelin (synthetic peptide analogs of LH-RH) Other antineoplastic protocols include the use of an inhibitor compound with another treatment modality, e.g., surgery or radiation, also referred to herein as “adjunct anti-neoplastic modalities.”

Exemplary therapeutic agents suitable for inclusion in the controlled-release formulations of the present invention include but are not limited to synthetic antibacterial agents of hardly water-soluble pyridone-carboxylic acid type such as benofloxacin, nalidixic acid, enoxacin, ofloxacin, amifloxacin, flumequine, tosfloxacin, piromidic acid, pipemidic acid, miloxacin, oxolinic acid, cinoxacin, norfloxacin, ciprofloxacin, pefloxacin, lomefloxacin, enrofloxacin, danofloxacin, binfloxacin, sarafloxacin, ibafloxacin, difloxacin and salts thereof. Other therapeutic agents include penicillin, tetracycline, cephalosporins and other antibiotics, antibacterial substances, antihistamines and decongestants, anti-inflammatories, antiparasitics, antivirals, local anesthetics, antifungal, amoebicidal, or trichomonocidal agents, analgesics, antiarthritics, antiasthmatics, anticoagulants, anticonvulsants, antidepressants, antidiabetics, antineoplastics, antipsychotics, antihypertensives and muscle relaxants. Representative antibacterial substances are beta-lactam antibiotics, tetracyclines, chloramphenicol, neomycin, gramicidin, bacitracin, sulfonamides, nitrofurazone, nalidixic acid and analogs and the antimicrobial combination of fludalanine/pentizidone. Representative antihistamines and decongestants are perilamine, chlorpheniramine, tetrahydrozoline and antazoline.

Anti-inflammatory drugs can also be used in combination with the 4-phenylbutyric acid controlled-release formulations. Representative anti-inflammatory drugs suitable for use include but are not limited to cortisone, hydrocortisone, betamethasone, dexamethasone, fluocortolone, prednisolone, triamcinolone, indomethacin, sulindac and its salts and corresponding sulfide. A representative antiparasitic compound is ivermectin.

Representative antiviral compounds are acyclovir and interferon. Representative analgesic drugs are diflunisal, aspirin or acetaminophen. Representative antiarthritics are phenylbutazone, indomethacin, silindac, its salts and corresponding sulfide, dexamethasone, ibuprofen, allopurinol, oxyphenbutazone or probenecid. Representative antiasthma drugs are theophylline, ephedrine, beclomethasone dipropionate and epinephrine. Representative anticoagulants are bishydroxycoumarin, and warfarin. Representative anticonvulsants are diphenylhydantoin and diazepam. Representative antidepressants are amitriptyline, chlordiazepoxide perphenazine, protriptyline, imipramine and doxepin. Representative antidiabetics are insulin, somatostatin and its analogs, tolbutamide, tolazamide, acetohexamide and chlorpropamide. Representative antineoplastics are adriamycin, fluorouracil, methotrexate and asparaginase. Representative antipsychotics are prochlorperazine, thioridazine, molindone, fluphenazine, trifluoperazine, perphenazine, armitriptyline and trifluopromazine. Representative antihypertensives are spironolactone, methyldopa, hydralazine, clonidine, chlorothiazide, deserpidine, timolol, propranolol, metoprolol, prazosin hydrochloride and reserpine. Representative muscle relaxants are succinylcholine-chloride, danbrolene, cyclobenzaprine, methocarbamol and diazepam.

Some other examples of therapeutic agents which can be included in the controlled-release formulations of the present invention include, but are not limited to, adiphenine, allobarbital, aminobenzoic acid, amobarbital, ampicillin, anethole, aspirin, azopropazone, azulene barbituric acid, beclomethasone, beclomethasone dipropronate, bencyclane, benzaldehyde, benzocaine, benzodiazepines, benzothiazide, betamethasone, betamethasone 17-valerate, bromobenzoic acid, bromoisovalerylurea, butyl-p-aminobenzoate, chloralhydrate, chlorambucil, chloramphenicol, chlorobenzoic acid, chlorpromazine, cinnamic acid, clofibrate, coenzyme A, cortisone, cortisone acetate, cyclobarbital, cyclohexyl anthranilate, deoxycholic acid, dexamethasone, dexamethasone acetate, diazepam, digitoxin, digoxin, estradiol, flufenamic acid, fluocinolone acetonide, 5-fluorouracil, flurbiprofen, griseofulvin, guaiazulene, hydrocortisone, hydrocortisone acetate, ibuprofen, indican, indomethacin, iodine, ketoprofen, lankacidin-group antibiotics, mefenamic acid, menadione, mephobarbital, metharbital, methicillin, metronidazole, mitomycin, nitrazepam, nitroglycerin, nitrosureas, paramethasone, penicillin, pentobarbital, phenobarbital, phenobarbitone, phenyl-butyric acid, phenyl-valeric acid, phenyloin, prednisolone, prednisolone acetate, progesterone, propylparaben, proscillaridin, prostaglandin A series, prostaglandin B series, prostaglandin E series, prostaglandin F series, quinolone antimicrobials, reserpine, spironolactone, sulfacetamide sodium, sulfonamide, testosterone, thalidomide, thiamine dilaurylsulphate, thiamphenicolpalmitate, thiopental, triamcinolone, VIAGRA™, vitamin A, vitamin D-3 (cholecalciferol), vitamin E, vitamin K-3 (menadione), and warfarin.

In a particular embodiment, the controlled release formulations of the present invention can be used in combination or alternation with therapeutic agents used to treat prostate disease. Such therapeutic agents are also suitable for inclusion in the controlled release formulations of the present invention. Such agents include, but are not limited to, chemotherapeutic agents, luteinizing hormone releasing hormone (LH-RH) agonists and anti-androgen agents.

In another particular embodiment, the controlled release formulations of the present invention can be used in combination or alternation with therapeutic agents used to treat spinal muscular atrophy. Such therapeutic agents are also suitable for inclusion in the controlled release formulations of the present invention. Such agents include, but are not limited to, valproic acid, suberoylanilide hydroxamic acid, hydroxyurea, aclarubicin, quanzolines, tetracycline derivatives, aminoglycosides, indoprofen, creatine, riluzole and carnitine.

In a further embodiment, the controlled release formulations of the present invention can be used in combination or alternation with therapeutic agents used to treat amyotrophic lateral sclerosis. Such therapeutic agents are also suitable for inclusion in the controlled release formulations of the present invention. Such agents include, but are not limited to, 2-amino-6-(trifluoromethoxy)benzothiazole, including tamoxifen, thalidomide, AVP-923-Neurodex (Avanir Pharmaceuticals), Minocycline, buspirone, ritonavir and hydroxyurea, AEOL 10150 (Aeolus Pharmaceuticals), co-enzyme Q, and ceftriaxome, creatine, myotrophin (Cephalon), celebrex, neotrofin (NeoTherapeutics, Inc.), NAALADase (Guilford Pharmaceuticals), oxandrolone, Topiramate (Topamax), Xaliproden (Sanofi-Synthelabo Inc), Indinavir and creatine.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Production of Slow Release Table of Sodium 4-Phenylbutyrate

A mixture of 6.000 Kg of sodium 4-phenylbutyrate (Triple Crown America, Inc., Perkasie, PA), 6.280 Kg of lactosum monohydricum, 3.500 Kg of Methocel K100 M Premium (Prochem AG, Zürich, Switzerland), and 750 g of Avicel PH 102 (Select Chemie, Zürich, Switzerland) was stirred in a Diosna Mixer (DIOSNA Dierks & Sohne GmbH, Osnabruck, Germany) and then wettened with 4,000.0 g of aqua purificata (water purified by inversion osmosis), and dried in cold air over the course of 18 hours. The mixture was then forced through a sieve IV mm, and dried again over the course of 10 hours with 40° C. air flow in a Lükon drying cabinet (Lükon Thermal Solutions AG, Tauffelen, Switzerland). A mixture of 240.0 of talcum and 30.0 g of magnesium stearate was then admixed over the course of 20 minutes. The resultant mixture was then pressed into 0.70 g tablets (using a Korsch tablet press EK II from Korsch AG, Berlin, Germany), having a thickness of about 6.8 mm and a hardness of about 90 Newton. This batch produced 24,000 tablet cores.

The cores were provided with a film coating using a colloidal dispersion containing 7,850.0 g of isopropyl alcohol, 3,360.0 g of Eudragit™ L 12.5, 66.0 g of dibutyl phthalate, 18.0 g of Miglyol 812, and 56.0 g of polyethylene glycol PEG 400. The suspension was sprayed at 3.5 atü and 25° C. onto the 24,000 tablet cores from above. The resultant film-coated tablets were dried in a circulating air-drying cabinet (Lükon) for at least 4 hours at 35° C.

All of the compositions, methods, and/or processes disclosed and claimed herein can be made and executed without undue experimentation in light of the present disclosure. While the compositions and methods of this invention have been described in terms of preferred embodiments, it will be apparent to those of skill in the art that variations may be applied to the compositions, methods and/or processes and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

1. A controlled-release composition comprising a therapeutically effective amount of 4-phenylbutyric acid, or pharmaceutically acceptable salts, esters or prodrugs thereof, dispersed in a polymer matrix, wherein the polymer matrix comprises a co-polymer, terpolymer or polymer blend.

2. The controlled-release composition of claim 1, wherein the polymer matrix comprises a co-polymer.

3. The controlled-release composition of claim 2, wherein the co-polymer comprises hydroxypropylmethylcellulose.

4. The controlled-release composition of claim 1, wherein the polymer matrix comprises a terpolymer.

5. The controlled-release composition of claim 4, wherein the terpolymer comprises hydroxypropylmethylcellulose.

6. The controlled-release composition of claim 1, wherein the polymer matrix comprises a polymer blend.

7. The controlled-release composition of claim 6, wherein the polymer blend comprises at least one hydrophilic polymer.

8. The controlled-release composition of claim 7, wherein the hydrophilic polymer is hydroxpropylmethylcellulose.

9. The controlled-release composition of claim 7, wherein the polymer blend comprises two or more hydrophilic polymers.

10. The controlled release composition of claim 9, wherein the polymer blend comprises hydroxypropylmethylcellulose and a cellulose ether selected from the group consisting of methylcellulose, hydroxyethylcellulose, hydroxymethylcellulose, carboxymethylcellulose, ethylcellulose, hydroxpropylcellulose and microcrystalline cellulose.

11. The controlled release composition of claim 7, further comprising a hydrophobic polymer.

12. The controlled release composition of claim 1, further comprising a therapeutically effective amount of a second therapeutic agent.

13. The controlled release composition of claim 12, wherein the second therapeutic agent is a chemotherapeutic agent.

14. The controlled release composition of claim 13, wherein the chemotherapeutic agent is selected from the group consisting of alkyating agents, antimetabolites, plant alkaloids, topoisomerase inhibitors, anti-tumor antibiotics and hormonal agents.

15. The controlled release composition of claim 12, wherein the second therapeutic agent is 2-amino-6-(trifluoromethoxy) benzothiazole.

16. The controlled release composition of claim 12, wherein the second therapeutic agent is selected from the group consisting of valproic acid, suberoylanilide hydroxamic acid, hydroxyurea, aclarubicin, quanzolines, tetracycline derivatives, aminoglycosides, indoprofen, creatine, riluzole and carnitine.

17. The controlled release composition of claim 1, wherein the 4-phenylbutyric acid salt is sodium phenylbutyrate.

18. A controlled release composition comprising a therapeutically effective amount of 4-phenylbutyric acid, or a pharmaceutically acceptable salt, ester or prodrug thereof, dispersed in a polymer matrix, said polymer matrix comprising at least one cellulose ether polymer selected from the group consisting of methylcellulose, hydroxyethyl cellulose and hydroxypropyl cellulose.

19. The controlled release composition of claim 18, further comprising a second therapeutic agent.

20. The controlled release composition of claim 18, wherein the 4-phenylbutyric acid salt is sodium phenylbutryate.

21. A controlled release composition comprising a therapeutically effective amount of 4-phenylbutyric acid, or a pharmaceutically acceptable salt, ester or prodrug thereof, dispersed in a polymer matrix, said polymer matrix comprising at least one hydrophilic polymer selected from the group consisting of non-cellulose polysaccharides, polyethylene oxide, polyvinyl alcohols and acrylic acid co-polymers.

22. The controlled release composition of claim 21, further comprising a second therapeutic agent.

23. The controlled release composition of claim 21, wherein the 4-phenylbutyric acid salt is sodium phenylbutyrate.

24. A method of treating a disease or disorder, comprising administering to a subject in need thereof a controlled release composition comprising a therapeutically effective amount of 4-phenylbutyric acid, or a pharmaceutically acceptable salt, ester or prodrug thereof, dispersed in a polymer matrix, wherein the polymer matrix comprises a co-polymer, terpolymer or polymer blend.

25. The method of claim 24, wherein the subject is human.

26. The method of claim 24, wherein the polymer matrix comprises a polymer blend.

27. The method of claim 26, wherein the polymer blend comprises hydroxypropylmethylcellulose.

28. The method of claim 24, wherein the disease or disorder is a neoplastic disorder.

29. The method of claim 28, wherein the disease or disorder is prostate cancer.

30. The method of claim 24, wherein the disease or disorder is a neurodegenerative disease or disorder.

31. The method of claim 30, wherein the neurodegenerative disease or disorder is spinal muscular atrophy.

32. The method of claim 30, wherein the neurodegenerative disease or disorder is amyotrophic lateral sclerosis

33. The method of claim 24, wherein the disease or disorder is selected from the group consisting of urea cycle disorders, hematological disorders, infectious diseases, cystic fibrosis, and protein localization or aggregation disorders.

34. The method of claim 24, wherein the 4-phenylbutyric acid salt is sodium phenylbutyrate.

35. A method of treating a disease or disorder, comprising administering to a subject in need thereof a controlled release composition comprising a therapeutically effective amount of 4-phenylbutyric acid, or a pharmaceutically acceptable salt, ester or prodrug thereof, dispersed in a hydrophilic polymer matrix, wherein the hydrophilic polymer is a cellulose derivative selected from the group consisting of methylcellulose, hydroxyethyl cellulose, hydroxypropyl cellulose, carboxymethylcellulose, hydroxomethylcellulose, hemicellulose, and methylcellulose.

36. The method of claim 35, wherein the subject is human.

37. The method of claim 35, wherein the disease or disorder is a neoplastic disorder.

38. The method of claim 35, wherein the neoplastic disorder is prostate cancer.

39. The method of claim 35, wherein the disorder is a neurodegenerative disease or disorder.

40. The method of claim 39, wherein the neurodegenerative disease or disorder is spinal muscular atrophy.

41. The method of claim 39, wherein the neurodegenerative disease or disorder is amyotrophic lateral sclerosis

42. The method of claim 35, wherein the disease or disorder is selected from the group consisting of urea cycle disorders, hematological disorders, infectious diseases, cystic fibrosis, and protein localization or aggregation disorders.

43. The method of claim 35, wherein the 4-phenylbutyric acid salt is sodium phenylbutyrate.

44. A method of treating a disease or disorder, comprising administering to a subject in need thereof a controlled release composition comprising a therapeutically effective amount of 4-phenylbutyric acid, or a pharmaceutically acceptable salt, ester or prodrug thereof, dispersed in a hydrophilic polymer matrix, wherein the hydrophilic polymer is selected from the group consisting of non-cellulose polysaccharides, polyethylene oxide, polyvinyl alcohols and acrylic acid co-polymers.

45. The method of claim 44, wherein the subject is human.

46. The method of claim 44, wherein the disease or disorder is a neoplastic disease or disorder.

47. The method of claim 46, wherein the neoplastic disease or disorder is prostate cancer.

48. The method of claim 44, wherein the disease or disorder is a neurodegenerative disease or disorder.

49. The method of claim 48, wherein the neurodegenerative disease or disorder is spinal muscular atrophy.

50. The method of claim 49, wherein the neurodegenerative disease or disorder is amyotrophic lateral sclerosis

51. The method of claim 44, wherein the disease or disorder is selected from the group consisting of urea cycle disorders, hematological disorders, infectious diseases, cystic fibrosis, and protein localization or aggregation disorders.

52. The method of claim 44, wherein the 4-phenylbutyric acid salt is sodium phenylbutyrate.

53. A method of treating spinal muscular atrophy by administering to a subject in need thereof a controlled release composition comprising a therapeutically effective amount of 4-phenylbutyric acid, or a pharmaceutically acceptable salt, ester or prodrug thereof, dispersed in a polymer matrix.

54. The method of claim 53, wherein the polymer matrix comprises at least one hydrophilic polymer.

55. The method of claim 54, wherein the hydrophilic polymer is selected from the group consisting of cellulose derivatives, non-cellulose polysaccharides, polyethylene oxide, polyvinyl alcohols and acrylic acid co-polymers.

56. The method of claim 55, wherein the cellulose derivative is a cellulose ether selected from the group consisting of methylcellulose, hydroxpropylmethylcellulose, hydroxyethyl cellulose and hydroxylpropyl cellulose.

57. The method of claim 56, wherein the cellulose derivative is hydroxypropylmethylcellulose.

58. The method of claim 53, wherein the controlled release composition further comprises a second therapeutic agent.

59. The method of claim 58, wherein the second therapeutic agent is selected from the group consisting of valproic acid, suberoylanilide hydroxamic acid, hydroxyurea, aclarubicin, quanzolines, tetracycline derivatives, aminoglycosides, indoprofen, creatine, riluzole and carnitine.

60. The method of claim 53, wherein the 4-phenylbutyric acid salt is sodium phenylbutyrate.

61. The method of claim 53, wherein the controlled release composition is administered orally.

62. A method of treating amyotrophic lateral sclerosis by administering to a subject in need thereof a controlled release composition comprising a therapeutically effective amount of 4-phenylbutyric acid, or a pharmaceutically acceptable salt, ester or prodrug thereof, dispersed in a polymer matrix.

63. The method of claim 62, wherein the polymer matrix comprises at least one hydrophilic polymer.

64. The method of claim 63, wherein the hydrophilic polymer is selected from the group consisting of cellulose derivatives, non-cellulose polysaccharides, polyethylene oxide, polyvinyl alcohols and acrylic acid co-polymers.

65. The method of claim 64, wherein the cellulose derivative is a cellulose ether selected from the group consisting of methylcellulose, hydroxpropylmethylcellulose, hydroxyethyl cellulose and hydroxylpropyl cellulose.

66. The method of claim 65, wherein the cellulose ether is hydroxypropylmethylcellulose.

67. The method of claim 62, wherein the controlled release composition further comprises a second therapeutic agent.

68. The method of claim 67, wherein the second therapeutic agent is 2-amino-6-(trifluoromethoxy) benzothiazole.

69. The method of claim 62, wherein the controlled release composition is administered orally.

70. The method of claim 62, wherein the 4-phenylbutyric acid salt is sodium phenylbutyrate.

71. A method of treating a neoplastic disease or disorder, comprising administering to a subject in need thereof a controlled release composition comprising a therapeutically effective amount of 4-phenylbutyric acid, or a pharmaceutically acceptable salt, ester or prodrug thereof, dispersed in a polymer matrix, wherein the polymer matrix comprises a co-polymer, terpolymer or polymer blend.

72. The method of claim 71, wherein the subject is human.

73. The method of claim 71, wherein the polymer matrix comprises a polymer blend.

74. The method of claim 73, wherein the polymer blend comprises hydroxypropylmethylcellulose.

75. The method of claim 71, wherein the neoplastic disease or disorder is prostate cancer.

76. The method of claim 71, wherein the controlled release composition further comprises a second therapeutic agent.

77. The method of claim 76, wherein the second therapeutic agent is selected from the group consisting of chemotheraputic agents, luteinizing hormone-releasing hormone (LH-RH) agonists and anti-androgen agonists.

78. The method of claim 71, wherein the 4-phenylbutyric acid salt is sodium phenylbutyrate.

79. A method of treating a neoplastic disease or disorder by administering to a subject in need thereof a controlled release composition comprising a therapeutically effective amount of 4-phenylbutyric acid, or a pharmaceutically acceptable salt, ester or prodrug thereof, dispersed in a hydrophilic polymer matrix, wherein the hydrophilic polymer is cellulose ether selected from the group consisting of methylcellulose, hydroxpropylmethylcellulose, hydroxyethyl cellulose and hydroxylpropyl cellulose.

80. The method of claim 79, wherein the subject is human.

81. The method of claim 79, wherein the neoplastic disease or disorder is prostate cancer.

82. The method of claim 79, wherein the controlled release composition further comprises a second therapeutic agent selected from the group consisting of chemotherapeutic agents, luteinizing hormone releasing hormone (LH-RH) agonists and anti-androgen agents.

83. The method of claim 79, wherein the 4-phenylbutyric acid salt is sodium phenylbutyrate.

84. A method of treating a neoplastic disease or disorder by administering to a subject in need thereof a controlled release composition comprising a therapeutically effective amount of 4-phenylbutyric acid, or a pharmaceutically acceptable salt, ester or prodrug thereof, dispersed in a hydrophilic polymer matrix, wherein the hydrophilic polymer is selected from the group consisting of non-cellulose polysaccharides, polyethylene oxide, polyvinyl alcohols and acrylic acid co-polymers

85. The method of claim 84, wherein the subject is human.

86. The method of claim 84, wherein the neoplastic disease is prostate cancer.

87. The method of claim 84, wherein the controlled release composition further comprises a second therapeutic agent selected from the group consisting of chemotherapeutic agents, luteinizing hormone releasing hormone (LH-RH) agonists and anti-androgen agents.

88. The method of claim 84, wherein the 4-phenylbutyric acid salt is sodium phenylbutyrate.
